SEQUENCE LISTING (16 sequences)

SEQ ID NO: 1 - protein, 40 aa, Artificial Sequence
  Aptide against STAT3 fused with nona-arginine
HGFQWPGSWTWENGKWTWKGAYQFLKGGGGSRRRRRRRRR

SEQ ID NO: 2 - protein, 26 aa, Artificial Sequence
  Aptide against STAT3
HGFQWPGSWTWENGKWTWKGAYQFLK

SEQ ID NO: 3 - protein, 9 aa, Artificial Sequence
  nona-arginine
RRRRRRRRR

SEQ ID NO: 4 - protein, 12 aa, Artificial Sequence
  Tat peptide
GRKKRRQRRRPQ

SEQ ID NO: 5 - protein, 11 aa, Artificial Sequence
  SPACE
ACTGSTQHQCG

SEQ ID NO: 6 - protein, 11 aa, Artificial Sequence
  TD-1
ACSSSPSKHCG

SEQ ID NO: 7 - protein, 11 aa, Artificial Sequence
  DLP
ACKTGSHNQCG

SEQ ID NO: 8 - protein, 12 aa, Artificial Sequence
  LP-12
HIITDPNMAEYL

SEQ ID NO: 9 - protein, 26 aa, Artificial Sequence
  control aptide
HASDRNGSWTWENGKWTWKGLHEQSD

SEQ ID NO: 10 - protein, 26 aa, Artificial Sequence
  Aptide against VEGF
AAPTSFGSWTWENGKWTWKGWQMWHR

SEQ ID NO: 11 - DNA, 20 bp, Artificial Sequence
  Col1a1_forward
taggccattgtgtatgcagc

SEQ ID NO: 12 - DNA, 21 bp, Artificial Sequence
  Col1a1_reverse
acatgttcagctttgtggacc

SEQ ID NO: 13 - DNA, 21 bp, Artificial Sequence
  alpha-SMA_forward
gtcccagacatcagggagtaa

SEQ ID NO: 14 - DNA, 21 bp, Artificial Sequence
  alpha-SMA_reverse
tcggatacttcagcgtcagga

SEQ ID NO: 15 - DNA, 20 bp, Artificial Sequence
  GAPDH_forward
ttcaccaccatggagaaggc

SEQ ID NO: 16 - DNA, 20 bp, Artificial Sequence
  GAPDH_reverse
ggcatggactgtggtcatga

(19) United States
(12) Patent Application Publication
(10) Pub. No.: US 2020/0376138 A1
(43) Pub. Date: Dec. 3, 2020

(54) LIPID NANO PARTICLE COMPLEX COMPRISING APTIDE FUSED WITH CELL PENETRATING MATERIALS AND USE SAME

(71) Applicant: Korea Advanced Institute of Science and Technology, Daejeon (KR)

(72) Inventors: Sangyong Jon, Daejeon (KR); Jin Yong Kim, Daejeon (KR); Hyeongseop Keum, Daejeon (KR); Jinjoo Kim, Daejeon (KR)

(73) Assignee: Korea Advanced Institute of Science and Technology, Daejeon (KR)

(21) Appl. No.: 16/892,172

(22) Filed: Jun. 3, 2020

Related U.S. Application Data
(63) Continuation-in-part of application No. PCT/KR2019/006474, filed on May 30, 2019.

(51) Int. Cl. A61K 47/69; A61K 47/64; A61K 47/54; A61K 9/00; A61P 29/00; A61K 38/17; A61P 11/00

(52) U.S. Cl. CPC A61K 47/6929; A61K 47/64; A61K 47/544; A61P 11/00; A61P 29/00; A61K 9/007; A61K 38/1709; A61K 9/0014

(57) ABSTRACT

The present invention relates to a lipid nanoparticle complex comprising an aptide fused with a cell penetrating material and a use thereof. Particularly, the lipid nanoparticle complex according to the present invention contains long-chain and short-chain phospholipids, and comprises an aptide fused with a cell-penetrating material. In particular, when the long-chain and short-chain phospholipids are included in a specific molar ratio, the lipid nanoparticle complex exhibits a discoid structure. In addition, when the lipid nanoparticle complex comprises the aptide for STAT protein as an aptide, it has superior cell or skin cell permeability, delivers the aptide to the dermal layer to show treatment effect on psoriasis without side effects, inhibits the expression of fibrosis-related genes increased by TGF-β, and attenuates symptoms of pulmonary fibrosis in in vivo model. Thus, the lipid nanoparticle complex according to the present invention can be effectively used as a carrier for various aptides.

### CROSS REFERENCE TO RELATED APPLICATION

This application is a continuation-in-part of PCT/KR2019/006474, filed May 30, 2019, the entire contents of which are incorporated herein by reference.

### BACKGROUND

### 1. Field of the Invention

The present invention relates to a lipid nanoparticle complex comprising an aptide fused with a cell penetrating material and a use thereof.

### 2. Description of the Related Art

Recently, antibodies that are harmless to the human body and have a short drug development period have been developed as therapeutic agents. However, antibodies are recognized as foreign antigens in the body, cause side effects such as allergic reactions or hypersensitivity reactions, and the therapeutic agent using the antibody has a problem that the production cost is high. In addition, a topical application preparation using a targeted peptide drug such as antibodies has not been reported.

Therefore, the development of antibody alternative proteins has been started to solve the problems. The antibody alternative protein is a recombinant protein made to have a constant region and a variable region like an antibody, and is small in size and stable. The antibody alternative protein is prepared by selecting a protein having high specificity and affinity to a target material from the library prepared by substituting a certain part with amino acids of random sequence. In this regard, Korean Patent Publication No. 10-2015-0118252 discloses a cyclic β-hairpin based peptide binder capable of exhibiting its activity by fusing the peptide at both ends of the cyclic β-hairpin randomly and binding the peptide to a target molecule.

On the other hand, STAT3 protein is a transcription regulator that transmits signals of various types of cytokines and growth factors into the nucleus, and is essential for maintaining life.

In particular, it has been reported that STAT3 is overexpressed in keratinocytes of patients with psoriasis, psoriasis symptoms occur in transgenic mice over-expressing STAT3, and STAT3 protein inhibitors have an effect of alleviating the psoriasis symptoms in the psoriasis animal model. In addition, it has been reported that STAT3 protein is a key transcription factor in the differentiation, amplification and stabilization of Th17 cells associated with psoriasis etiology, as well as in the process of IL-17 secretion in the activated Th17 cells. Therefore, it has been found from the above that STAT3 protein is essential for the pathophysiology of psoriasis.

Psoriasis is a chronic inflammatory skin disease in which exacerbation and alleviation are repeated. Although the cause of psoriasis has not been clearly identified, it is generally known to be caused by an immunological abnormality in the body. When psoriasis develops, a little reddish rash appears on the skin, and white keratin (scalp) is covered on top of it, and in severe cases, the size of the rash expands to the size of a palm.

Psoriasis is a skin disease that can be seen anywhere in the world, although the incidence varies depending on race, ethnic group, and geographic location. It has a prevalence rate of around 3% worldwide. It is estimated that there are approximately 1.5 million people with psoriasis in Korea, similar to 3% of the population, and the prevalence rate is steadily increasing. Psoriasis can occur at all ages, but most are in their 20s, followed by those in their 10s and 30s.

General psoriasis treatment methods include local treatment, systemic treatment, and phototherapy. Recently, immunobiological agents based on the pathogenesis of psoriasis have been developed. In addition, in order to increase the therapeutic effect on psoriasis while reducing side effects, combination therapies using the above treatment methods suitably are widely used. Among them, topical treatments applied directly on the skin, such as ointments, lotions and gels, are essential treatments for psoriasis patients, and are the first and most widely used to control the symptoms of psoriasis. In particular, if you use a topical treatment, light psoriasis can have good effects without other treatments. In particularly, if the topical treatment is used well, light psoriasis can be treated efficiently without any other treatment. When the patient has other diseases such as digestive disorders and kidney disorders, it is safer and more effective to use an ointment than an oral drug. As topical treatments, vitamin D ointments, vitamin D complex gel preparations, steroid ointments, vitamin A ointments, and tar formulations are commercially available. In this regard, Korean Patent No. 10-1755407 discloses a skin external composition for treating psoriasis comprising a first pharmacologically active ingredient containing one or more vitamin D or vitamin D analogs, a second pharmacologically active ingredient containing one or more corticosteroids, and a non-aqueous solvent.

It is known that psoriasis is caused by abnormally overactivated interactions between keratinocytes and various immune cells. The keratinocytes stimulated by various endogenous or exogenous factors cause hyperproliferation and abnormal differentiation, resulting in shorter replacement cycles of keratinocytes. At this time, the secreted DNA-LL37 complex, RNA-LL37 complex, and the like induce the activation of various inflammatory cells such as dendritic cells and macrophages. The activated dendritic cells or macrophages secrete factors such as IL-1β, IL-6, IL-12, IL-23 and IL-36, and these factors induce the activation of T cells. In particular, IL-1β, IL-6, IL-12 and IL-23 induce the differentiation of Thl7 cells, the important factor of psoriasis etiology, and keratinocytes are stimulated again by IL-17 secreted from the activated Thl7 cells. This vicious cycle is repeated.

On the other hand, fibrosis means a phenomenon in which a part of an organ hardens for some reason, and renal fibrosis, pulmonary fibrosis and liver fibrosis are generally known. Renal fibrosis is caused by the prolonged duration of diseases such as glomerulonephritis, interstitial nephritis, diabetic nephropathy, etc. Patients with end-stage renal disease can only be treated by dialysis or kidney transplantation. Liver fibrosis is caused by prolonged duration of viral hepatitis, alcoholic hepatitis and fatty hepatitis. Liver fibrosis progresses in liver dysfunction or incidence of liver cancer. In the liver with progressed liver fibrosis, the incidence of liver failure or liver cancer is increased. Pulmonary fibrosis is caused by genetic or environmental factors. When pulmonary fibrosis is progressed, life expectancy is mostly less than 3 years, and can be treated only with lung transplantation.

As described above, fibrosis that may occur in various organs is progressed by secreting extracellular matrices such as collagen 1a1 (Col1a1), αSMA (α-smooth muscle actin) and fibronectin while fibroblasts are abnormally activated. In addition, the mechanism of TGF-β signaling is one of the most well-known mechanisms in the process of fibrosis.

Pulmonary fibrosis is an irremediable and life-threatening disease usually with a poor prognosis and characterized by their progressive architectural remodeling caused by lung parenchyma injury followed by an exaggerated would healing responses. Development of pulmonary fibrosis can lead to impaired lung functions typically accompanied by shortness of breath, ultimately resulting in death. Notorious for being involved with numerous risk factors in the development, the occurrence of pulmonary fibrosis is often considered idiopathic (IPF), thus repeatedly mismanaged. Presently, it is known that the average life expectancy following diagnosis is only 3-5 years. In terms of epidemiology, the current estimated prevalence of pulmonary fibrosis ranges from 10-60 cases per 100,000 person-years in the United States. Notably, there has been a prevalence report of 494 cases per 100,000 person-years in 2011, indicative of rapid prevalence increase in a decade.

Pathologically, various risk factors (smoking, micro-particle aspiration, aging, occupational hazards, and genetic factors) damage lung parenchyma. Stimulated alveolar macrophages secrete pro-inflammatory cytokines and chemokines suchlike interleukin-6 (IL-6), tumor necrosis factor-alpha (TNF-α), and interferon-gamma (IFN-γ), phosphorylating signal transducer and activator of transcription-3 (STAT3), which is a member of STAT protein family, triggering inflammation. STAT3 is activated through Janus kinase (JAK), SRC, c-Jun N-terminal kinases (JNK)-intermediated phosphorylation, triggering the STAT3 to form the dimer structures followed by translocation into the nucleus, where they are known to function as transcription factors. STAT3 activation initiates the secretion of chemokine ligand 2 and 3 (CCL2 and CCL3) recruiting macrophages, which later polarize into pro-fibrotic M2-type macrophages. M2 macrophages secretes local fibrotic mediators like, transforming growth factor-beta (TGF-β), and platelet-derived growth factor (PDGF) and these cytokines stimulate epithelial cells and residing fibroblasts to differentiate into myofibroblasts through epithelial-mesenchymal transition (EMT) causing extravagant extracellular matrix (ECM) accumulation in the lung resulting in aberrant repair modeling. Markedly, previous researches showed that STAT3-deficient fibroblasts were less subtle to the fibrotic effects of TGF-β and pharmacological inhibition of STAT3 phosphorylation successfully ameliorated fibrosis in animal models. This suggests that STAT3 is a core checkpoint in fibrosis signaling and STAT3 inhibitor might be a potent therapeutics candidate for pulmonary fibrosis.

In present, both pharmacological and non-pharmacological treatments are available for pulmonary fibrosis. Two FDA-approved medications, nintedanib (Growth factor receptor tyrosine kinase inhibitor), and pirfenidone (TGF-β inhibitor) demonstrated a momentous reduction in the forced vital capacity (FVC) decline rate. However, in addition to high-priced medication cost ($100,000/year), notable side effects of elevated inflammatory liver enzymes, gastrointestinal disorders, anorexia, and nausea can cause serious complications. Typical non-pharmacological approach, lung transplantation is another treatment option for the patients, but only a selected minority of patients can receive transplantation due to the limited organ supply and a cost of transplantation ranging at $500,000-$800,000. Besides, it should be noted that the 5-year survival rate of lung transplantation is only roughly 53%. Collectively, pulmonary fibrosis patients are in desperate need for safer, approachable, and potent alternative therapeutic measures.

Recently, using phage display screening and our aptide platform technology, we identified the specific STAT3 activation inhibiting aptide with high affinity (APTstat3) (HGFQWPGSWTWENGKWTWKGAYQFLK, SEQ ID NO: 2) and for improved cell penetration, poly-arginine cell-penetrating motif (9-arginine) was conjugated to form APTstat3-9R(HGFQWPGSWTWENGKWTWKGAYQFLKGGGGSRRRRRRRRR, SEQ ID NO: 1).

In previous studies, we observed the therapeutic efficacy of STAT3-inhibiting peptide against xenograft cancer and psoriasis-like inflammation models where STAT3 is constitutively activated.

Intratracheal instillation delivery is an encouraging route for approaching lung diseases because it is non-invasive with little systemic adverse effects. However, the nature of residing pulmonary surfactant in the lung surface makes it difficult for the uptake of many nanoparticles. Formed by type 2 pneumocyte, pulmonary surfactant is an indispensable structure of surface-active lipoprotein complex, which functions by reducing the alveolar surface tension thus lowering the required energy to inflate the lungs and plays innate host defense. In many previous reports, there have been numerous attempts to facilitate the therapeutic particles through the surfactant layer and it was demonstrated that lipid surface decorated nanoparticles were the most efficient in expedited delivery into the lung parenchyma due to lipophilic interaction between pulmonary surfactant and nanoparticle surface coated lipids.

In efforts to develop a carrier capable of stably and economically delivering the aptide for STAT3 protein, the present inventors prepared a lipid nanoparticle complex comprising the aptide for STAT3 protein fused with nonaarginine or choline, a cell penetrating material, and confirmed that the nanoparticle complex has skin permeability and skin cell permeability, and not only improves inflammation caused by psoriasis in a psoriasis animal model, but also expresses the expression of a fibrosis related gene induced by TGF-β in a fibroblast cell line, and reduced the signs of pulmonary fibrosis via inhibition of STAT3 activation and suppressed the expression of distinct immunological markers, resulting in the completion of the present invention.

### SUMMARY

It is an object of the present invention to provide a lipid nanoparticle complex comprising an aptide fused with a cell penetrating material and a use thereof

To achieve the above objects, the present invention provides a lipid nanoparticle complex comprising an aptide fused with a cell penetrating material.

The present invention also provides a method for preventing or treating inflammatory skin disease comprising the lipid nanoparticle complex according to the present invention as an active ingredient.

The present invention also provides a skin external application comprising the lipid nanoparticle complex according to the present invention as an active ingredient for the prevention or treatment of inflammatory skin disease.

The present invention also provides a cosmetic composition comprising the lipid nanoparticle complex according to the present invention as an active ingredient for the prevention or amelioration of inflammatory skin disease.

The present invention also provides a method for preventing or treating fibrosis comprising the lipid nanoparticle complex according to the present invention as an active ingredient.

In addition, the present invention provides a health functional food comprising the lipid nanoparticle complex according to the present invention as an active ingredient for the prevention or amelioration of fibrosis.

### BRIEF DESCRIPTION OF THE DRAWINGS

FIG. 1 is a set of transmission electron photomicrographs showing the structure according to the mixing molar ratio of DMPC and DHPC constituting the nanoparticle complex in the lipid nanoparticle complex in which the aptide for STAT3 protein fused with nonaarginine prepared in one embodiment of the present invention is included or not included.

FIGS. 2a and 2b are transmission electron photomicrographs showing the structure of the lipid nanoparticle complex comprising the aptide for STAT3 protein fused with cholic acid prepared in one embodiment of the present invention (FIG. 2a), and the structure of the lipid nanoparticle complex comprising the aptide for VEGF protein fused with nonaarginine (FIG. 2b).

FIGS. 3 and 4 are graphs showing the hydrodynamic diameter according to the mixing molar ratio of DMPC and DHPC constituting the nanoparticle complex in the lipid nanoparticle complex in which the aptide for STAT3 protein fused with nonaarginine prepared in one embodiment of the present invention is included or not included.

FIG. 5 is a graph showing the surface charge of the lipid nanoparticle complex in which the aptide for STAT3 protein fused with nonaarginine prepared in one embodiment of the present invention is included or not included.

FIG. 6 is a set of photomicrographs showing that the lipid nanoparticle complex in which the aptide for STAT3 protein fused with nonaarginine prepared in one embodiment of the present invention is included ([FITC-APT]-LNCs) has skin permeability in a psoriasis animal model unlike the aptide fusant for STAT3 protein fused with nonaarginine (FITC-APT), confirmed by confocal microscopy 1, 6 or 12 hours after application.

FIG. 7 is a set of photomicrographs showing that the lipid nanoparticle complex in which the aptide for STAT3 protein fused with nonaarginine prepared in one embodiment of the present invention is included ([FITC-APT]-LNCs) has skin permeability in a normal animal model unlike the aptide fusant for STAT3 protein fused with nonaarginine (FITC-APT), confirmed by two-photon microscopy.

FIG. 8 is a set of photomicrographs showing that the lipid nanoparticle complex in which the aptide for STAT3 protein fused with nonaarginine prepared in one embodiment of the present invention is included ([FITC-APT]-LNCs) has skin permeability in a psoriasis animal model unlike the aptide fusant for STAT3 protein fused with nonaarginine (FITC-APT), confirmed by two-photon microscopy at the depths of 6, 12, 18, 24, 30, 36 and 42 m from the skin epidermis.

FIG. 9 is a set of diagrams showing that the lipid nanoparticle complex in which the aptide for STAT3 protein fused with nonaarginine prepared in one embodiment of the present invention is included ([FITC-APT]-LNCs) has skin permeability in a psoriasis animal model unlike the aptide fusant for STAT3 protein fused with nonaarginine (FITC-APT), confirmed by observation of the skin cross section.

FIG. 10 is a graph showing the transmitted depth according to the relative fluorescence intensity measured from the lipid nanoparticle complex in which the aptide for STAT3 protein fused with nonaarginine prepared in one embodiment of the present invention is included ([FITC-APT]-LNCs) and the aptide fusant for STAT3 protein fused with nonaarginine (FITC-APT).

FIG. 11 is a set of photographs showing the skin cell permeability according to the treatment concentration of the lipid nanoparticle complex comprising the aptide for STAT3 protein fused with nonaarginine prepared in one embodiment of the present invention ([FITC-APT]-LNCs).

FIG. 12 is a set of graphs showing the skin cell permeability according to the treatment time of the lipid nanoparticle complex comprising the aptide for STAT3 protein fused with nonaarginine prepared in one embodiment of the present invention ([FITC-APT]-LNCs).

FIG. 13 is a set of graphs showing that the lipid nanoparticle complex comprising the aptide for STAT3 protein fused with nonaarginine prepared in one embodiment of the present invention penetrates HaCaT and NIH3T3 cells and is located in the cytoplasm.

FIG. 14 is a schematic diagram showing the experimental schedule for confirming the inflammatory effect using the lipid nanoparticle complex comprising the aptide for STAT3 protein fused with nonaarginine prepared in one embodiment of the present invention in a psoriasis animal model.

FIGS. 15a-15c are graphs showing the changes of the ear thickness (FIG. 15a), the PASI score (FIG. 15b) and the ear punch biopsy weight (FIG. 15c) by the lipid nanoparticle complex comprising the aptide for STAT3 protein fused with nonaarginine prepared in one embodiment of the present invention in a psoriasis animal model (Control: Vaseline application group, IMQ+DW: IMQ and DW application group, IMQ+LNCs: IMQ and the nanoparticle complex of Comparative Example 3 application group, IMQ+[FITC-APT]-LNCs: IMQ and the nanoparticle complex of Example 3 application group, IMQ+CLQ: IMQ and CLQ application group).

FIG. 16 is a set of photographs showing the therapeutic effect of the lipid nanoparticle complex comprising the aptide for STAT3 protein fused with nonaarginine prepared in one embodiment of the present invention on psoriasis (Gross), and the results of H&E staining (H&E) (IMQ: IMQ application group, Control: Vaseline application group, DW: DW application group, LNCs: the nanoparticle complex of Comparative Example 3 application group, [APTstat3-9R]-LNCs: the nanoparticle complex of Example 3 application group, CLQ: CLQ application group).

FIGS. 17a and 17b are graphs showing the changes of the epidermal thickness (FIG. 17a; IMQ: IMQ application group, Ctrl: Vaseline application group, DW: DW application group, LNCs: nanoparticle complex application group of Comparative Example 3, [APT]-LNCs: nanoparticle complex application group of Example 3, CLQ: CLQ application group) and the cytokine production in the tissue (FIG. 17b; Control: Vaseline application group, IMQ+DW: IMQ and DW application group, IMQ+LNCs: IMQ and the nanoparticle complex of Comparative Example 3 application group, IMQ+[APT]-LNCs: IMQ and the nanoparticle complex application group, IMQ+CLQ: IMQ and CLQ application group) by the lipid nanoparticle complex comprising the aptide for STAT3 protein fused with nonaarginine prepared in one embodiment of the present invention in a psoriasis animal model.

FIGS. 18a and 18b are graphs showing that the lipid nanoparticle complex comprising the aptide for STAT3 protein fused with nonaarginine prepared in one embodiment of the present invention does not affect the spleen size (FIG. 18a) and weight (FIG. 18b) in a psoriasis animal model (Control: Vaseline application group, IMQ+DW: IMQ and DW application group, IMQ+LNCs: IMQ and the nanoparticle complex of Comparative Example 3 application group, IMQ+[FITC-APT]-LNCs: IMQ and the nanoparticle complex of Example 3 application group, IMQ+CLQ: IMQ and CLQ application group).

FIG. 19 is a set of graphs showing the results of real-time PCR confirming that the Col1a1 or αSMA gene expression increased by TGF-β in the fibroblast cell line is suppressed by the lipid nanoparticle complex comprising the aptide for STAT3 protein fused with nonaarginine prepared in one embodiment of the present invention.

FIGS. 20a and 20b are diagrams showing the results of confirming that the lipid nanoparticle complex comprising the aptide for STAT3 protein fused with nonaarginine prepared in one embodiment of the present invention does not form aggregates even after the preparation (FIG. 20a), and maintains a disk shape of about 30 nm (FIG. 20b).

FIG. 21a is a diagram showing the results of confirming that the lipid nanoparticle complex comprising the aptide for STAT3 protein fused with nonaarginine suppresses M2 macrophage polarization by analyzing an expression of CD163 which is a M2 macrophage specific marker.

FIG. 21b is a diagram showing the results of confirming that the lipid nanoparticle complex comprising the aptide for STAT3 protein fused with nonaarginine suppresses myoblast differentiation by analyzing an expression of vimentin which is a mesenchymal filament marker.

FIG. 22a is a graph showing the results of confirming that the lipid nanoparticle complex comprising the aptide for STAT3 protein fused with nonaarginine increases pulmonary surfactant infiltration capability in lung tissues of bleomycin-induced pulmonary fibrosis mice model.

FIG. 22b is a graph showing the results of confirming that the lipid nanoparticle complex comprising the aptide for STAT3 protein fused with nonaarginine increases in penetration into MLE12 lung epithelial cells.

FIG. 23a is a scheme of administration of the lipid nanoparticle complex comprising the aptide for STAT3 protein fused with nonaarginine in bleomycin-induced pulmonary fibrosis mice model.

FIGS. 23b and 23c are graphs of showing the body weight (FIG. 23b) and body weight changes (FIG. 23c) in control group, bleomycin and PBS administrating group, bleomycin and control aptide administrating group and bleomycin and the lipid nanoparticle administrating group in fibrosis mice model.

FIG. 23d is graph of showing the lung weight in control group, bleomycin and PBS administrating group, bleomycin and control aptide administrating group and bleomycin and the lipid nanoparticle administrating group in fibrosis mice model.

FIG. 23e is graph of showing the lung/body weight ratio in control group, bleomycin and PBS administrating group, bleomycin and control aptide administrating group and bleomycin and the lipid nanoparticle administrating group in fibrosis mice model.

FIG. 23f is a graph of showing the lung wet-to-dry ratio in control group, bleomycin and PBS administrating group, bleomycin and control aptide administrating group and bleomycin and the lipid nanoparticle administrating group in fibrosis mice model.

FIG. 24a is a H&E staining picture of showing that minimal to moderate alveolar and bronchiole wall thickening, mildly maintained tissue density and infiltrated inflammatory foci were observed in the lipid nanoparticle complex comprising the aptide for STAT3 protein fused with nonaarginine administrating group.

FIG. 24b is a Sirius red & Fast green staining picture of showing that very small amount of collagen deposition was observed in the lipid nanoparticle complex comprising the aptide for STAT3 protein fused with nonaarginine administrating group.

FIG. 24c is a graph for calculating the severity of the disease in the results of FIG. 24a.

FIG. 24d is a graph of for calculating the collagen amount in the results of FIG. 24b.

FIG. 25a is a picture of IHC staining analysis to visualize distinct pulmonary fibrosis markers(CD163, pSTAT3, CD20 and Mast cell).

FIG. 25b-25e are graphs showing the % expression of distinct pulmonary fibrosis markers(e.g. CD163(FIG. 25b), pSTAT3(FIG. 25c), CD20(FIG. 25d) and Mast cell(FIG. 25e)), respectively.

FIG. 26a is a graph of showing the spleen size and weight in control group, bleomycin and PBS administrating group, bleomycin and control aptide administrating group and bleomycin and the lipid nanoparticle administrating group in fibrosis mice model.

FIG. 26b-26g are graphs showing the change of the indicators of systemic hepatotoxicity(Aspartate aminotransferase (AST)(FIG. 26b), Alanine aminotransferase (ALT)(FIG. 26c), AST/ALT ratio(FIG. 26d), Glucose (GLU)(FIG. 26e), Blood urea nitrogen (BUN)(FIG. 26f), and Creatinine (CRE)(FIG. 26g), respectively).

### SEQUENCE LISTING

The nucleic and amino acid sequences listing in the accompanying sequence listing are shown using standard letter abbreviations for nucleotide bases, and three letter code for amino acids, as defined in 37 C.F.R. 1.822. The Sequence Listing is submitted as an ASCII text file in the form of the file named “Sequence.txt” (˜4.00 kb), which was created on Jun. 3, 2020, and which is incorporated by reference herein.

### DETAILED DESCRIPTION

Hereinafter, the present invention is described in detail.

The present invention provides a lipid nanoparticle complex comprising an aptide fused with a cell penetrating material.

The term “aptide” used in this specification refers to an aptamer-like peptide with improved stability while maintaining affinity for the target. The said aptide consists of a scaffold comprising a cyclic β-hairpin-based peptide binder and n amino acids at both ends of the scaffold, which can bind to a specific biological target. The aptide can include a target-binding region capable of constructing various libraries. The aptide can be composed of any one or more amino acids selected from the group consisting of L-amino acids and D-amino acids. The term “stability” may include physical, chemical and biological stability of the aptide, and specifically may mean biological stability. That is, the biologically stable aptide can be resistant to the action of protease in the body.

The aptide according to the present invention can be an aptide that specifically binds to STAT3 (signal transducer and activator of transcription 3) protein, and can specifically be composed of the amino acid sequence represented by SEQ. ID. NO: 2. the aptide for STAT3 protein is stable with a size of about 5 kDa, and can bind to STAT3 protein with a high affinity of 200 nM. The aptide for STAT3 protein can inhibit the activation of STAT3 protein by binding to SH2 domain of STAT3 protein and suppressing the phosphorylation. This mechanism may have less effect on an inaccurate target than a conventional JAK inhibitor that inhibits the activation of STAT3 protein using the JAK inhibitory mechanism.

The aptide can be a variant or fragment of an aptide having a different sequence by deletion, insertion, substitution or a combination of amino acid residues within a range that does not affect the structure and activity of the aptide according to the present invention. Amino acid exchanges in proteins or peptides that do not entirely alter the activity of a molecule are well informed in the art. In some cases, it can be modified by phosphorylation, sulfation, acrylication, saccharification, methylation and farnesylation. The aptide can have 70, 80, 85, 90, 95 or 98% homology to the amino acid sequence represented by SEQ. ID. NO: 2.

The lipid nanoparticle complex according to the present invention can be composed of phospholipids. The term “phospholipid” is a kind of compound lipid and refers to a general term for lipids comprising phosphate ester. The phospholipid can include a hydrophilic region composed of phosphatidylcoline and a hydrophobic region composed of fatty acid, and can be classified into long-chain and short-chain phospholipids according to the length of the fatty acid, the hydrophobic region. The lipid nanoparticle complex can include long-chain and short-chain phospholipids, particularly, the long-chain and short-chain phospholipids can be included in a molar ratio of 0.5˜7:1, 0.5˜5:1, 0.5˜4:1, 1˜7:1, 1˜5:1, 1˜4:1, 2˜7:1, 2˜5:1 or 2˜4:1. The lipid nanoparticle complex can have a diameter of 10 to 500 nm, 10 to 450 nm, 10 to 400 nm, 20 to 400 nm, 30 to 350 nm, 40 to 300 nm, 50 to 250 nm, 60 nm to 200 nm, 100 to 200 nm, 150 to 250 nm, 200 to 300 nm, 250 to 350 nm, 300 to 400 nm, 350 to 450 nm, 400 to 500 nm, 10 to 350 nm, 10 to 300 nm, 10 to 250 nm, 10 to 200 nm, 10 to 150 nm, 10 to 100 nm, 15 to 100 nm, 20 to 100 nm, 10 to 80 nm, 15 to 80 nm, 20 to 80 nm, 10 to 60 nm, 15 to 60 nm, 20 to 60 nm, 10 to 50 nm, 15 to 50 nm, 20 to 50 nm, 10 to 40 nm, 15 to 40 nm, 20 to 40 nm, 10 to 35 nm, 15 to 35 nm or 20 to 35 nm.

In addition, the long-chain and short-chain phospholipids can be appropriately selected and applied by those skilled in the art, and can include both long-chain or short-chain phospholipids known in the art. The long-chain and short-chain phospholipids can be modified, and specifically, can be labeled with PEG (polyethylene glycol). The PEG can include all PEGs known in the art, which can be specifically PEG500, PEG2000, and the like. In addition, the long-chain and short-chain phospholipids can be labeled with fluorescent materials. The fluorescent material can include all fluorescent materials known in the art, which can be PE (phycoerythrin) and FITC (fluorescein isothiocyanate). Furthermore, the long-chain and short-chain phospholipids according to the present invention can be labeled with gadolinium. The lipid nanoparticle complex labeled with gadolinium of the present invention can be used as a MRI contrast medium.

The long-chain phospholipid can be any one or more selected from the group consisting of 1,2-dilauroyl-sn-glycero-3-phosphocholine (12:0 PC, DLPC), 1,2-ditridecanoyl-sn-glycero-3-phophocholine (13:0 PC), 1,2-dimyristoyl-sn-glycero-3-phosphocholine (14:0 PC, DMPC), 1,2-dipentadecanoyl-sn-glycero-3-phophocholine (15:0 PC), 1,2-dipalmitoyl-sn-glycero-3-phosphocholine (16:0 PC, DPPC), 1,2-diphytanoyl-sn-glycero-3-phosphocholine (4ME 16:0 PC), 1,2-diheptadecanoyl-sn-glycero-3-phophocholine (17:0 PC), 1,2-distearoyl-sn-glycero-3-phosphocholine (18:0 PC, DSPC), 1,2-dinonadecanoyl-sn-glycero-3-phophocholine (19:0 PC), 1,2-diarachidoyl-sn-glycero-phosphocholine (20:0 PC), 1,2-dihenarachidoyl-sn-glycero-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-palmitoyl-2-oleoyl-5,w-glycero-3-phosphocholine (POPC) and 1-palmitoyl-2-stearoyl-5,w-glycero-3-phosphocholine (PSPC). Meanwhile, the short-chain phospholipid can be any one or more selected from the group consisting of 1,2-dipropionyl-sn-glycero-3-phosphocholine (3:0 PC), 1,2-dibutyryl-sn-glycero-3-phosphocholine (4:0 PC), 1,2-dipentanoyl-sn-glycero-3-phosphocholine (5:0 PC), 1,2-dihexanoyl-sn-glycero-3-phosphocholine (6:0 PC, DHPC), 1,2-diheptanoyl-sn-glycero-3-phosphocholine (7:0 PC, DHPC) and 1,2-dioctanoyl-sn-glycero-3-phosphocholine (8:0 PC).

The lipid nanoparticle complex according to the present invention can exhibit a discoid structure. The lipid nanoparticle complex having the discoid structure can pass through a narrow gap between keratinocytes constituting the skin, and can have an advantage that the migration thereof between the cell layers is faster than that of spherical particles. In addition, the lipid nanoparticle complex having the discoid structure has excellent surface adhesiveness, and can accelerate the penetration rate in the skin by increasing the lipid fluidity in the stratum corneum of the skin.

The cell penetrating material is a substance that permeates the cells, and can include any material known in the art. For example, the cell penetrating material can be any one or more selected from the group consisting of peptides and compounds. The aptide fused with the material above can be included in the lipid nanoparticle complex according to the present invention at the concentration of 0.2 to 30 weight %, 1 to 30 weight %, 5 to 30 weight %, 0.2 to 25 weight %, 1 to 25 weight %, 5 to 25 weight %, 0.2 to 20 weight %, 1 to 20 weight %, 5 to 20 weight %, 0.2 to 15 weight %, 1 to 15 weight % or 5 to 15 weight % by the total weight of the complex.

Particularly, the peptide can be any one or more peptides selected from the group consisting of polyarginine, Tat, SPACE (skin penetration and cell entering peptide), TD-1 (transdermal peptide-1), DLP (dermis localizing peptide) and LP-12 (linear peptide-12 mer). For example, the polyarginine can be a peptide composed of the amino acid sequence represented by SEQ. ID. NO: 3, Tat can be a peptide composed of the amino acid sequence represented by SEQ. ID. NO: 4, SPACE can be a peptide composed of the amino acid sequence represented by SEQ. ID. NO: 5, TD-1 can be a peptide composed of the amino acid sequence represented by SEQ. ID. NO: 6, DLP can be a peptide composed of the amino acid sequence represented by SEQ. ID. NO: 7, and LP-12 can be a peptide composed of the amino acid sequence represented by SEQ. ID. NO: 8. On the other hand, the compound can be any one or more selected from the group consisting of cholic acid, oleic acid and derivatives thereof. The compound can be appropriately modified by a person skilled in the art as long as it maintains cell permeability.

In a preferred embodiment of the present invention, the present inventors prepared a nanoparticle complex comprising the long-chain and short-chain phospholipids in a molar ratio of 1:1, 2:1 or 3:1 by thin-film hydration. Particularly, DMPC was used as the long-chain phospholipid, DHPC was used as the short-chain phospholipid, and the mixture of DMPC and DHPC was hydrated using a solution containing the aptide for STAT3 protein fused with nonaarginine, thereby a lipid nanoparticle complex comprising the aptide for STAT3 protein fused with a cell-permeable peptide.

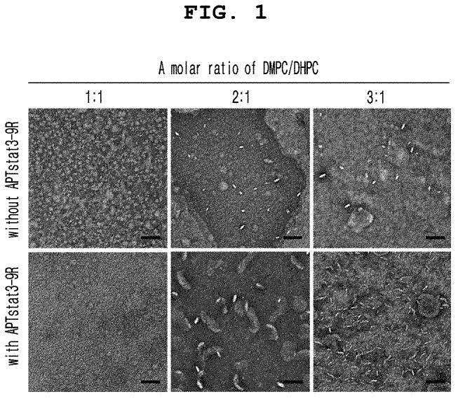

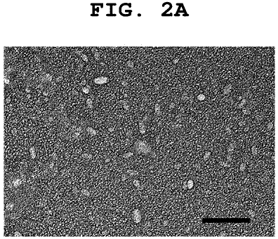

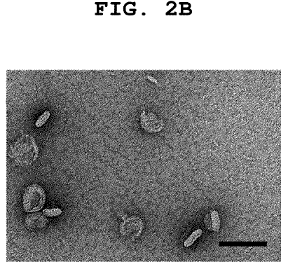

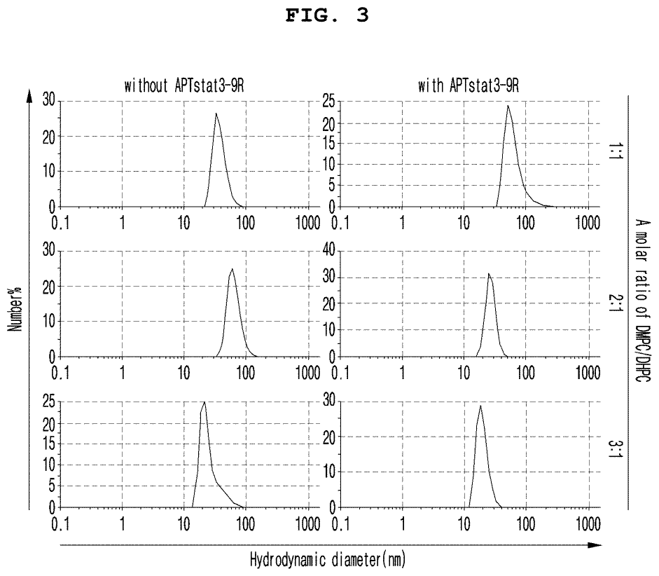

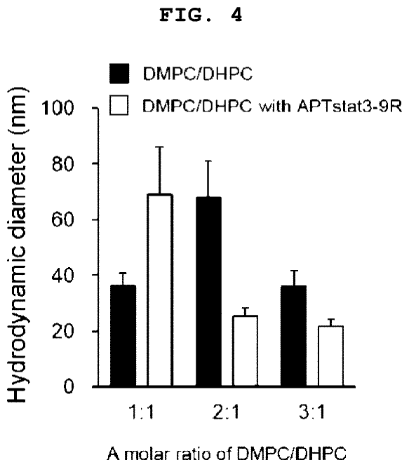

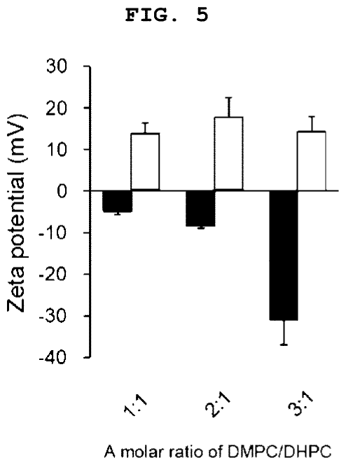

It was confirmed that the nanoparticle complex showed a discoid structure as the molar ratio of the long-chain and short-chain phospholipids used in the preparation of the nanoparticle complex increased, in particular, the nanoparticle complex containing the long-chain and short-chain phospholipids in a molar ratio of 3:1 showed a clear discoid structure by measuring the particle size and zeta potential of the prepared nanoparticle complex (see FIG. 1). In addition, the average diameter and thickness of the nanoparticle complex confirmed under a transmission electron microscope were 31.3±6.8 nm and 7.7±1.4 nm, respectively, and the hydrodynamic diameter thereof was about 20 to 30 nm (see FIGS. 3 and 4), and the surface was positively charged (see FIG. 5). When cholic acid was used instead of the cell-permeable peptide, or when the aptide for VEGF protein was used instead of the aptide for STAT3 protein, a stable discoid nanoparticle complex having a diameter of about 30 nm was formed (see FIGS. 2a and 2b).

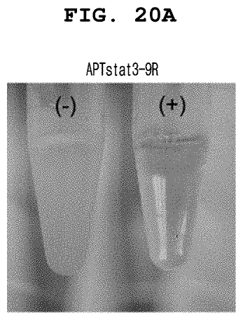

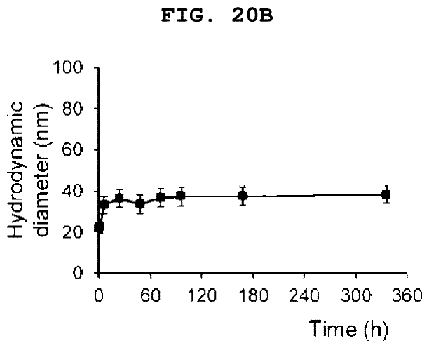

In addition, the lipid nanoparticle complex maintained as a stable discoid nanoparticle complex of uniform size without forming aggregates for a long time after production (FIGS. 20a and 20b).

From the above results, it was confirmed that a discoid lipid nanoparticle complex comprising an aptide fused with a cell penetrating material was prepared.

The present invention also provides a method for treating or ameliorating inflammatory skin disease comprising a step of administering the lipid nanoparticle complex of claim 1 to a subject in need thereof.

The inflammatory skin disease can be STAT3-related inflammatory skin disease, and the STAT3-related inflammatory skin disease includes one or more of psoriasis or atopy.

For example, the lipid nanoparticle complex can be an aptide fused with a cell penetrating material, and specifically, the aptide can be an aptide specifically binding to STAT3 protein. At this time, the aptide that specifically binds to STAT3 protein can be a substance exhibiting physiological activity in the pharmaceutical composition according to the present invention. On the other hand, the cell penetrating material can include any material known to penetrate cells in the art.

In addition, the lipid nanoparticle complex can include long-chain and short-chain phospholipids. The long-chain and short-chain phospholipids can be included in a molar ratio of 0.5˜7:1, 0.5˜5:1, 0.5˜4:1, 1˜7:1, 1˜5:1, 1˜4:1, 2˜7:1, 2˜5:1 or 2˜4:1. The long-chain and short-chain phospholipids can be appropriately selected and applied by those skilled in the art. Particularly, the long-chain phospholipid can be any one or more selected from the group consisting of 1,2-dilauroyl-sn-glycero-3-phosphocholine, 1,2-ditridecanoyl-sn-glycero-3-phophocholine, 1,2-dimyristoyl-sn-glycero-3-phosphocholine, 1,2-dipentadecanoyl-sn-glycero-3-phophocholine, 1,2-dipalmitoyl-sn-glycero-3-phosphocholine, 1,2-diphytanoyl-sn-glycero-3-phosphocholine, 1,2-diheptadecanoyl-sn-glycero-3-phophocholine, 1,2-distearoyl-sn-glycero-3-phosphocholine, 1,2-dinonadecanoyl-sn-glycero-3-phophocholine, 1,2-diarachidoyl-sn-glycero-phosphocholine, 1,2-dihenarachidoyl-sn-glycero-phosphocholine, 1,2-dibehenoyl-sn-glycero-3-phosphocholine, 1,2-ditricosanoyl-sn-glycero-3-phosphocholine, 1,2-dilignoceroyl-sn-glycero-3-phosphocholine, 1-palmitoyl-2-oleoyl-sn-glycero-3-phosphocholine and 1-palmitoyl-2-stearoyl-sn-glycero-3-phosphocholine. Meanwhile, the short-chain phospholipid can be any one or more selected from the group consisting of 1,2-dipropionyl-sn-glycero-3-phosphocholine, 1,2-dibutyryl-sn-glycero-3-phosphocholine, 1,2-dipentanoyl-sn-glycero-3-phosphocholine, 1,2-dihexanoyl-sn-glycero-3-phosphocholine, 1,2-diheptanoyl-sn-glycero-3-phosphocholine and 1,2-dioctanoyl-sn-glycero-3-phosphocholine.

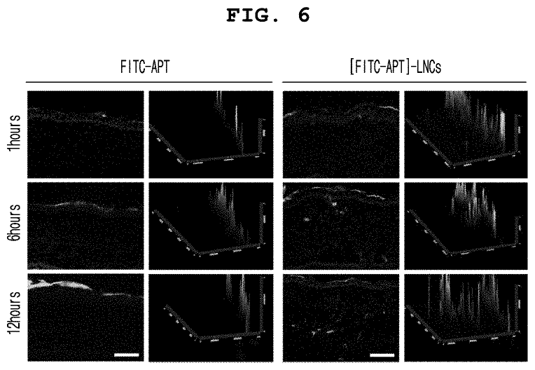

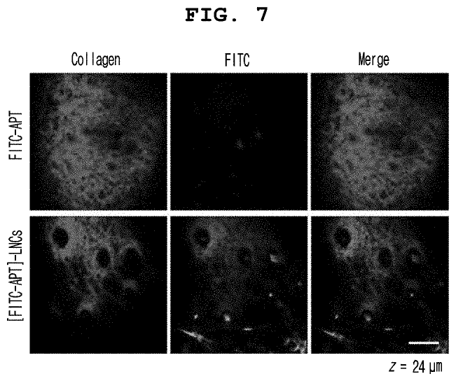

In a preferred embodiment of the present invention, the present inventors prepared a lipid nanoparticle complex comprising the aptide for STAT3 protein fused with nonaarginine, and confirmed that when the complex was applied on the skin, it transmitted the aptide, a bioactive substance, through the skin to the dermal layer using a confocal microscope (see FIG. 6) and a two-photon microscope (see FIGS. 7 to 10).

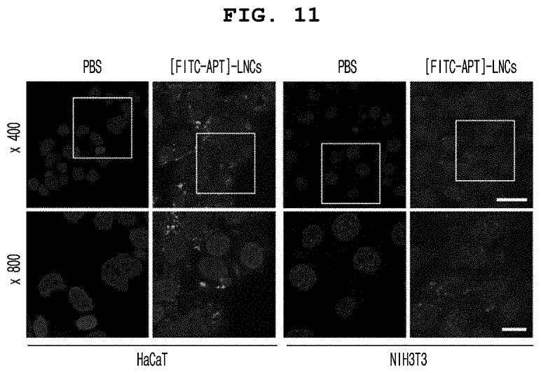

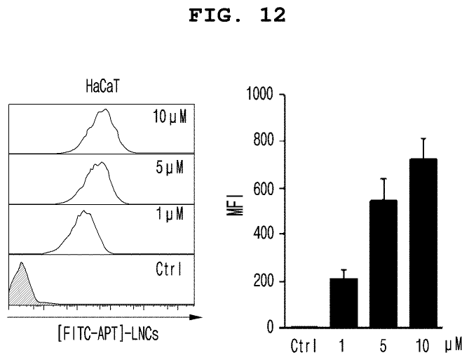

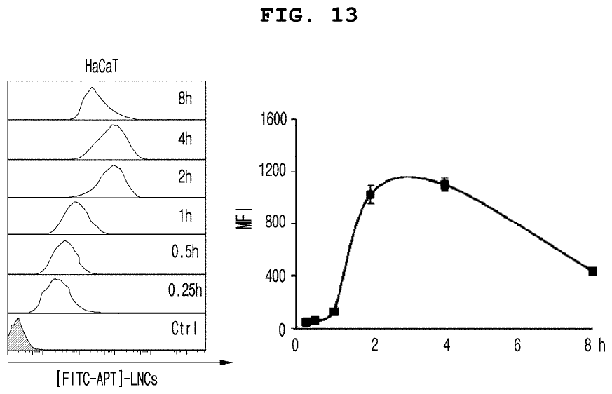

In addition, it was confirmed that the lipid nanoparticle complex increased skin cell permeability according to the treatment concentration and time (see FIGS. 11 and 12), and that the lipid nanoparticle complex treated on the cells was located in the cytoplasm (see FIG. 13).

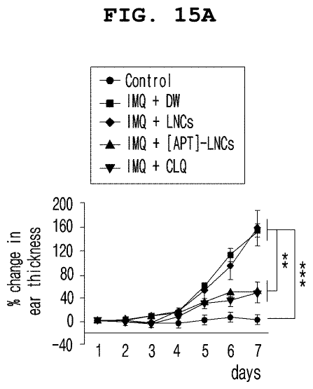

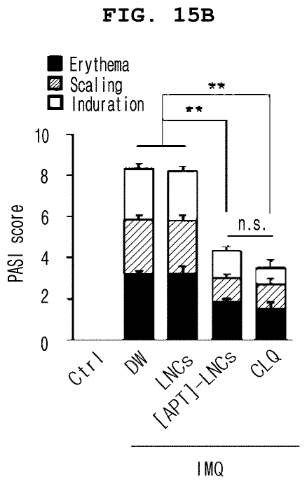

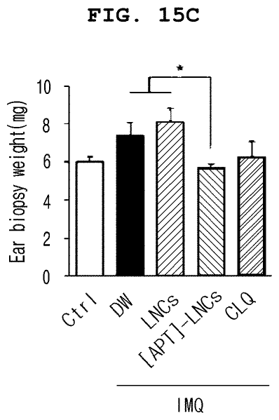

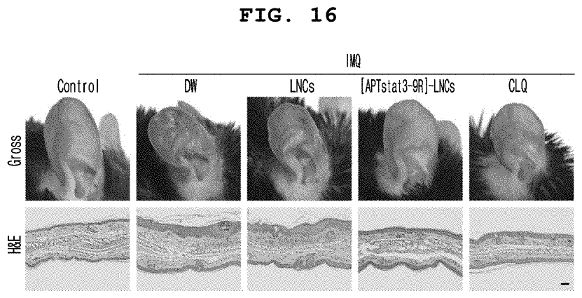

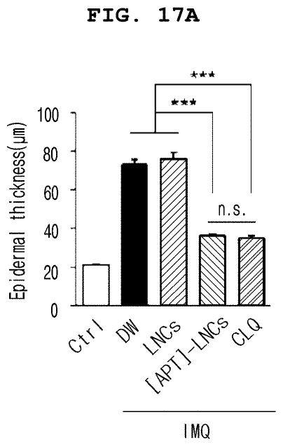

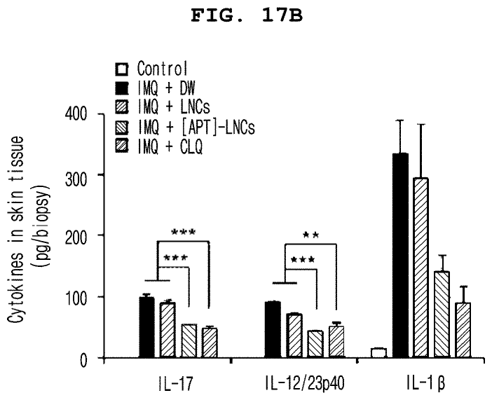

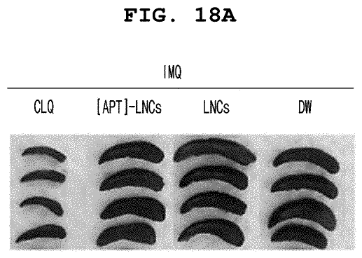

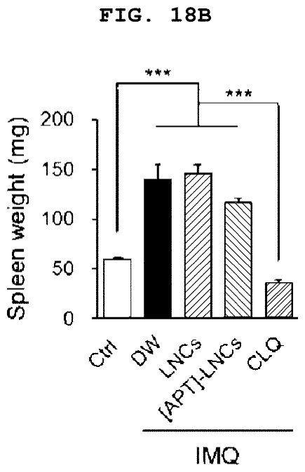

When the lipid nanoparticle complex according to the present invention was applied on the skin of a psoriasis-induced animal model, the ear thickness, the PASI score and the ear punch biopsy weight were significantly reduced (see FIGS. 15a to 15c). As a result of visual observation or H&E staining, it was confirmed that psoriasis was ameliorated in an animal model treated with the lipid nanoparticle complex (see FIG. 16). In addition, in the animal model applied with the lipid nanoparticle complex according to the present invention, epidermal hyperplasia and inflammatory cell infiltration were improved, (see FIG. 17a) and the productions of IL-17, IL-12/23p40 and IL-1β, the psoriasis-related cytokines, were significantly reduced (see FIG. 17b). On the other hand, the size and weight of the spleen of the animal model applied with the lipid nanoparticle complex according to the present invention were increased, but the spleen of the animal model applied with CLQ was shrivelled. It is presumed that the complex affected the body's immune system. It is presumed that the complex affected the systemic immune system (see FIGS. 18a and 18b).

Therefore, it was confirmed that the lipid nanoparticle complex according to the present invention can be effectively used for the prevention or treatment of inflammatory skin disease without side effects on the whole body.

The present invention also provides a method for treating or ameliorating inflammatory skin disease comprising a step of applying the lipid nanoparticle complex of claim 1 to a skin of a subject in need thereof.

The skin external application according to the present invention can include the lipid nanoparticle complex described above as an active ingredient. For example, the lipid nanoparticle complex can be an aptide fused with a cell penetrating material, and specifically, the aptide can be an aptide specifically binding to STAT3 protein. At this time, the aptide that specifically binds to STAT3 protein can be a substance exhibiting physiological activity in the skin external application according to the present invention. On the other hand, the cell penetrating material can include any material known to penetrate cells in the art.

In addition, the lipid nanoparticle complex can include long-chain and short-chain phospholipids. The long-chain and short-chain phospholipids can be included in a molar ratio of 0.5˜7:1, 0.5˜5:1, 0.5˜4:1, 1˜7:1, 1˜5:1, 1˜4:1, 2˜7:1, 2˜5:1 or 2˜4:1. The long-chain and short-chain phospholipids can be appropriately selected and applied by those skilled in the art. Particularly, the long-chain phospholipid can be any one or more selected from the group consisting of 1,2-dilauroyl-sn-glycero-3-phosphocholine, 1,2-ditridecanoyl-sn-glycero-3-phophocholine, 1,2-dimyristoyl-sn-glycero-3-phosphocholine, 1,2-dipentadecanoyl-sn-glycero-3-phophocholine, 1,2-dipalmitoyl-sn-glycero-3-phosphocholine, 1,2-diphytanoyl-sn-glycero-3-phosphocholine, 1,2-diheptadecanoyl-sn-glycero-3-phophocholine, 1,2-distearoyl-sn-glycero-3-phosphocholine, 1,2-dinonadecanoyl-sn-glycero-3-phophocholine, 1,2-diarachidoyl-sn-glycero-phosphocholine, 1,2-dihenarachidoyl-sn-glycero-phosphocholine, 1,2-dibehenoyl-sn-glycero-3-phosphocholine, 1,2-ditricosanoyl-sn-glycero-3-phosphocholine, 1,2-dilignoceroyl-sn-glycero-3-phosphocholine, 1-palmitoyl-2-oleoyl-sn-glycero-3-phosphocholine and 1-palmitoyl-2-stearoyl-sn-glycero-3-phosphocholine. Meanwhile, the short-chain phospholipid can be any one or more selected from the group consisting of 1,2-dipropionyl-sn-glycero-3-phosphocholine, 1,2-dibutyryl-sn-glycero-3-phosphocholine, 1,2-dipentanoyl-sn-glycero-3-phosphocholine, 1,2-dihexanoyl-sn-glycero-3-phosphocholine, 1,2-diheptanoyl-sn-glycero-3-phosphocholine and 1,2-dioctanoyl-sn-glycero-3-phosphocholine.

In a preferred embodiment of the present invention, the present inventors prepared a lipid nanoparticle complex comprising the aptide for STAT3 protein fused with nonaarginine, and confirmed that when the complex was applied on the skin, it transmitted the aptide, a bioactive substance, through the skin to the dermal layer using a confocal microscope (see FIG. 6) and a two-photon microscope (see FIGS. 7 to 10).

In addition, it was confirmed that the lipid nanoparticle complex increased skin cell permeability according to the treatment concentration and time (see FIGS. 11 and 12), and that the lipid nanoparticle complex treated on the cells was located in the cytoplasm (see FIG. 13).

When the lipid nanoparticle complex according to the present invention was applied on the skin of a psoriasis-induced animal model, the ear thickness, the PASI score and the ear punch biopsy weight were significantly reduced (see FIGS. 15a to 15c). As a result of visual observation or H&E staining, it was confirmed that psoriasis was ameliorated in an animal model treated with the lipid nanoparticle complex (see FIG. 16). In addition, in the animal model applied with the lipid nanoparticle complex according to the present invention, epidermal hyperplasia and inflammatory cell infiltration were improved, (see FIG. 17a) and the productions of IL-17, IL-12/23p40 and IL-1β, the psoriasis-related cytokines, were significantly reduced (see FIG. 17b). On the other hand, the size and weight of the spleen of the animal model applied with the lipid nanoparticle complex according to the present invention were increased, but the spleen of the animal model applied with CLQ was shrivelled. It is presumed that the complex affected the body's immune system. It is presumed that the complex affected the systemic immune system (see FIGS. 18a and 18b).

Therefore, it was confirmed that the lipid nanoparticle complex according to the present invention can be effectively used for the prevention or treatment of inflammatory skin disease without side effects on the whole body.

The skin external application of the present invention can include pharmaceutically acceptable carriers and excipients. The carrier and excipient can include preservatives, stabilizers, hydrating agents, emulsifying accelerators and buffers. Particularly, the excipient can be lactose, dextrin, starch, mannitol, sorbitol, glucose, saccharose, microcrystalline cellulose, gelatin, polyvinylpyrrolidone or a mixture thereof. The skin external application can be appropriately prepared according to the methods well known in the art. The skin external application can be prepared in the form of powder, gel, ointment, cream, liquid and aerosol.

The present invention also provides a method for treating or ameliorating fibrosis comprising a step of administering the lipid nanoparticle complex of claim 1 to a subject in need thereof.

The fibrosis is selected from the group consisting of pulmonary fibrosis, renal fibrosis and liver fibrosis.

The lipid nanoparticle complex is administered by an intratracheal instillation.

For example, the lipid nanoparticle complex can be an aptide fused with a cell penetrating material, and specifically, the aptide can be an aptide specifically binding to STAT3 protein. At this time, the aptide that specifically binds to STAT3 protein can be a substance exhibiting physiological activity in the pharmaceutical composition according to the present invention. On the other hand, the cell penetrating material can include any material known to penetrate cells in the art.

In addition, the lipid nanoparticle complex can include long-chain and short-chain phospholipids. The long-chain and short-chain phospholipids can be included in a molar ratio of 0.5˜7:1, 0.5˜5:1, 0.5˜4:1, 1˜7:1, 1˜5:1, 1˜4:1, 2˜7:1, 2˜5:1 or 2˜4:1. The long-chain and short-chain phospholipids can be appropriately selected and applied by those skilled in the art. Particularly, the long-chain phospholipid can be any one or more selected from the group consisting of 1,2-dilauroyl-sn-glycero-3-phosphocholine, 1,2-ditridecanoyl-sn-glycero-3-phophocholine, 1,2-dimyristoyl-sn-glycero-3-phosphocholine, 1,2-dipentadecanoyl-sn-glycero-3-phophocholine, 1,2-dipalmitoyl-sn-glycero-3-phosphocholine, 1,2-diphytanoyl-sn-glycero-3-phosphocholine, 1,2-diheptadecanoyl-sn-glycero-3-phophocholine, 1,2-distearoyl-sn-glycero-3-phosphocholine, 1,2-dinonadecanoyl-sn-glycero-3-phophocholine, 1,2-diarachidoyl-sn-glycero-phosphocholine, 1,2-dihenarachidoyl-sn-glycero-phosphocholine, 1,2-dibehenoyl-sn-glycero-3-phosphocholine, 1,2-ditricosanoyl-sn-glycero-3-phosphocholine, 1,2-dilignoceroyl-sn-glycero-3-phosphocholine, 1-palmitoyl-2-oleoyl-sn-glycero-3-phosphocholine and 1-palmitoyl-2-stearoyl-sn-glycero-3-phosphocholine. Meanwhile, the short-chain phospholipid can be any one or more selected from the group consisting of 1,2-dipropionyl-sn-glycero-3-phosphocholine, 1,2-dibutyryl-sn-glycero-3-phosphocholine, 1,2-dipentanoyl-sn-glycero-3-phosphocholine, 1,2-dihexanoyl-sn-glycero-3-phosphocholine, 1,2-diheptanoyl-sn-glycero-3-phosphocholine and 1,2-dioctanoyl-sn-glycero-3-phosphocholine.

The pharmaceutical composition according to the present invention can include the above mentioned formulation, dosage, administration method, and the like.

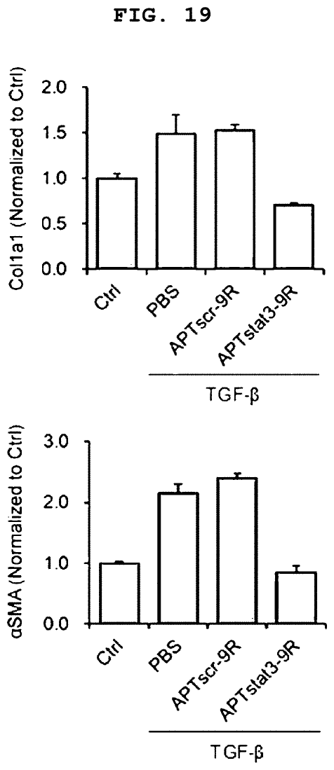

In a preferred embodiment of the present invention, the present inventors prepared a lipid nanoparticle complex comprising the aptide for STAT3 protein fused with nonaarginine, and confirmed that the lipid nanoparticle complex inhibited the expression of fibrosis-related genes increased by TGF-β (see FIG. 19).

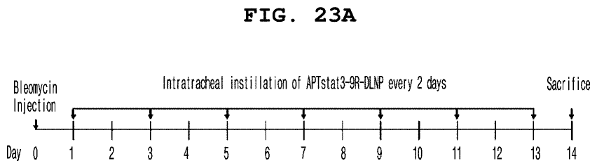

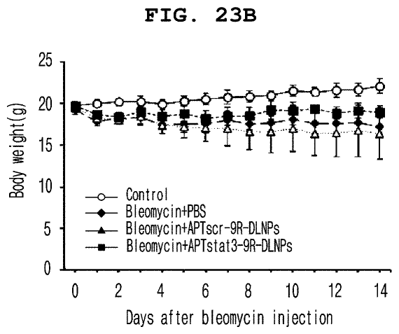

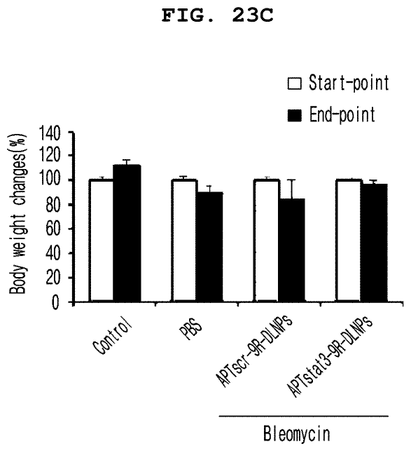

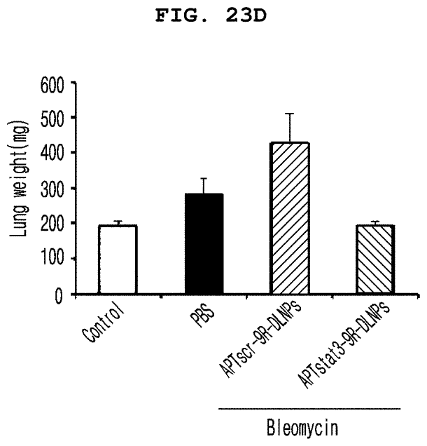

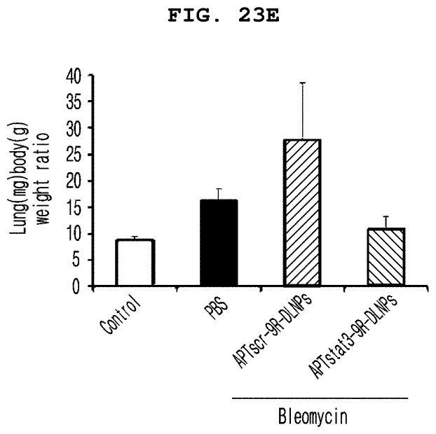

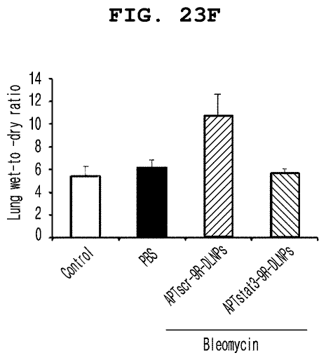

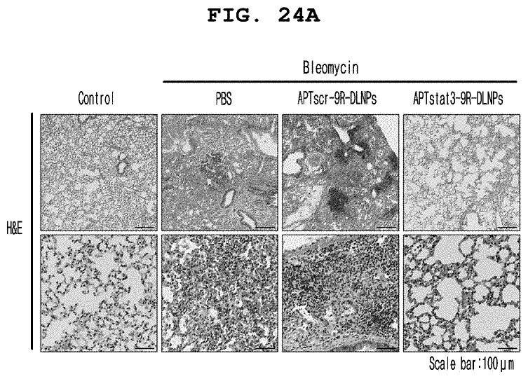

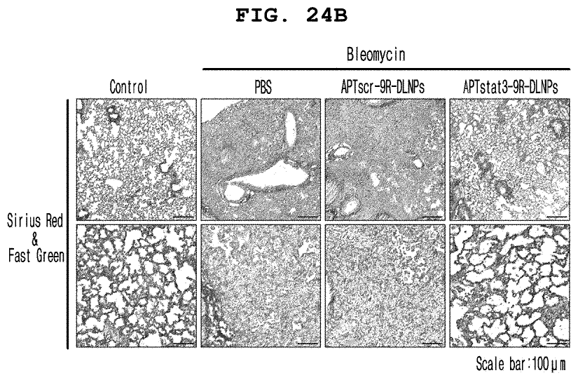

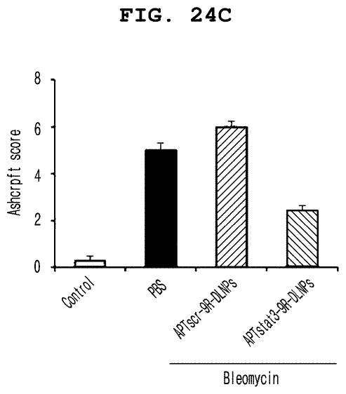

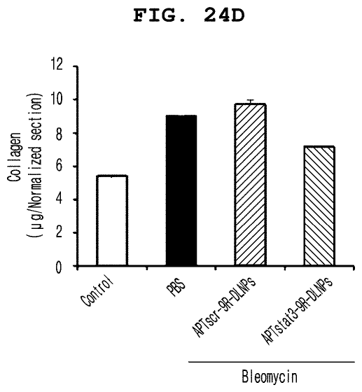

When the lipid nanoparticle complex according to the present invention was delivered by intratracheal instillation in bleomycin-induced pulmonary fibrosis mice model, the symptoms of pulmonary fibrosis were significantly reduced (see FIG. 23a-23f), and the severity of disease and collagen accumulation are also reduced (see FIG. 24a-24d).

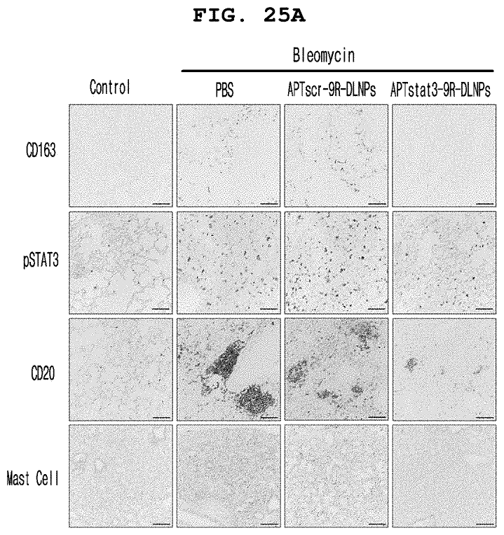

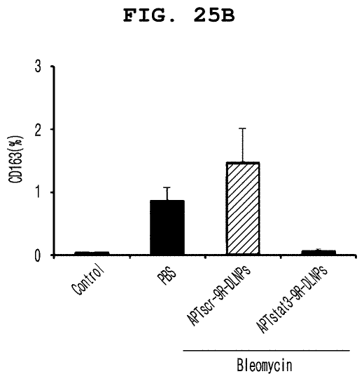

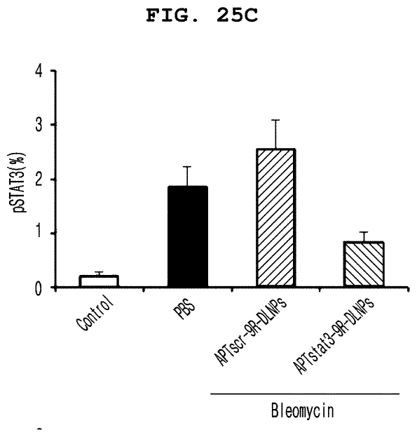

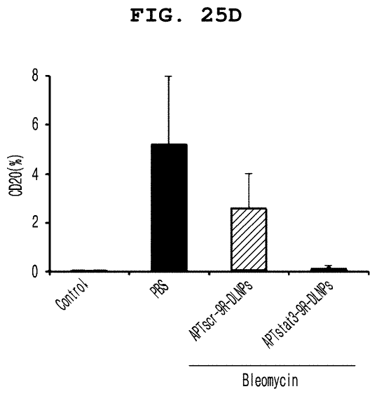

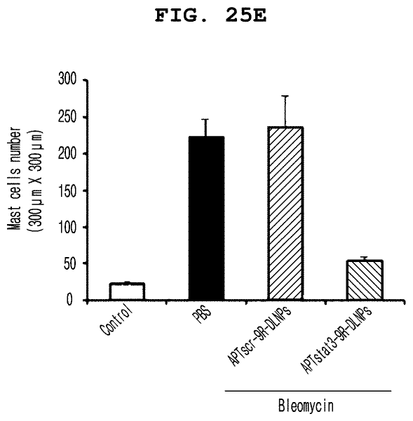

In addition, in the animal model administered with the lipid nanoparticle complex according to the present invention, the increased expression of pulmonary fibrosis markers were reduced (see FIG. 25a-25e).

Therefore, it was confirmed that the lipid nanoparticle complex according to the present invention can be effectively used for the prevention or treatment of fibrosis without side effects on the whole body.

In addition, the present invention provides a health functional food comprising the lipid nanoparticle complex according to the present invention as an active ingredient for the prevention or amelioration of fibrosis.

### Advantageous Effect

The lipid nanoparticle complex according to the present invention includes long chain and short chain phospholipids, while comprising an aptide fused with a cell penetrating material. In particular, when the long chain and short chain phospholipids are included in a specific molar ratio, the lipid nanoparticle complex exhibits a discoid structure. When the lipid nanoparticle complex comprises the aptide for STAT protein as an aptide, it has better cell or skin cell permeability compared to the case where only the aptide is included, delivers the aptide to the dermis to show the effect of treating psoriasis without side effects, and inhibits the expression of the fibrosis-related gene increased by TGF-β, and attenuates symptoms of pulmonary fibrosis in in vivo model. Therefore, the lipid nanoparticle complex according to the present invention can be effectively used as a carrier for various aptides.

### INDUSTRIAL APPLICABILITY

The lipid nanoparticle complex according to the present invention contains long-chain and short-chain phospholipids, and comprises an aptide fused with a cell-penetrating material. In particular, when the long-chain and short-chain phospholipids are included in a specific molar ratio, the lipid nanoparticle complex exhibits a discoid structure. In addition, when the lipid nanoparticle complex comprises the aptide for STAT protein as an aptide, it has superior cell or skin cell permeability, delivers the aptide to the dermal layer to show treatment effect on psoriasis without side effects, and inhibits the expression of fibrosis-related genes increased by TGF-β. Thus, the lipid nanoparticle complex according to the present invention can be effectively used as a carrier for various aptides.

Hereinafter, the present invention will be described in detail by the following examples.

However, the following examples are only for illustrating the present invention, and the contents of the present invention are not limited thereto.

### EXAMPLES

### Example 1: Preparation of Nanoparticle Complex Comprising Aptide for STAT3 Protein and Nonaarginine, and Long-Chain and Short-Chain Phospholipids in Molar Ratio of 1:1

A lipid nanoparticle complex comprising the aptide for STAT3 protein and a cell-permeable peptide was prepared by thin-film hydration. At this time, long-chain and short-chain phospholipids were mixed in a molar ratio of 1:1 to prepare the nanoparticle complex.

Particularly, the long-chain phospholipid DMPC (1,2-dimyristoyl-sn-glycero-3-phosphocholine) and the short-chain phospholipid DHPC (1,2-dihexanoyl-sn-glycero-3-phosphocholine) were dissolved in chloroform, respectively. The dissolved DMPC and DHPC were mixed in a molar ratio of 1:1, which was mixed by vortexing. The mixture was evaporated under nitrogen gas to obtain a thin lipid film. Meanwhile, the aptide (SEQ. ID. NO: 1) for STAT3 protein fused with nonaarginine, a cell-permeable peptide, was prepared by requesting Anigen (Korea). The obtained thin film was hydrated using the solution containing the aptide until the aptide became 10% (w/w) by the total lipid. Sonication was performed until the thin film hydrated at room temperature turned into a transparent solution. As a result, a lipid nanoparticle complex comprising the aptide for STAT3 protein fused with a cell-permeable peptide was prepared.

### Example 2: Preparation of Nanoparticle Complex Comprising Aptide for STAT3 Protein and Nonaarginine, and Long-Chain and Short-Chain Phospholipids in Molar Ratio of 2:1

A nanoparticle complex was prepared in the same conditions and methods as described in Example 1, except that DMPC and DHPC were mixed in a molar ratio of 2:1.

### Example 3: Preparation of Nanoparticle Complex Comprising Aptide for STAT3 Protein and Nonaarginine, and Long-Chain and Short-Chain Phospholipids in Molar Ratio of 3:1

A nanoparticle complex was prepared in the same conditions and methods as described in Example 1, except that DMPC and DHPC were mixed in a molar ratio of 3:1.

### Example 4: Preparation of Nanoparticle Complex Comprising Aptide for STAT3 Protein and Cholic Acid, and Long-Chain and Short-Chain Phospholipids in Molar Ratio of 3:1

A nanoparticle complex wherein the aptide for STAT3 protein was fused with cholic acid as a cell-penetrating material, and DMPC and DHPC were included in a molar ratio of 2:1 was prepared. The experiment was performed in the same conditions and methods as described in Example 1, except that cholic acid was added instead of the cell-permeable peptide.

### Example 5: Preparation of Nanoparticle Complex Comprising Aptide for VEGF Protein and Nonaarginine, and Long-Chain and Short-Chain Phospholipids in Molar Ratio of 3:1

A nanoparticle complex was prepared in the same conditions and methods as described in Example 3, except that the aptide (SEQ. ID. NO: 10) for VEGF protein fused with nonaarginine was used instead of the aptide for STAT3 protein fused with nonaarginine was used.

### Comparative Example 1: Preparation of Nanoparticle Complex not Comprising Aptide for STAT3 Protein and Cell-Permeable Peptide, but Comprising Long-Chain and Short-Chain Phospholipids in Molar Ratio of 1:1

A nanoparticle complex not comprising the aptide for STAT3 protein and the cell-permeable peptide, but comprising long-chain and short-chain phospholipids in a molar ratio of 1:1 was prepared in the same conditions and methods as described in Example 1, except that hydration was performed using distilled water instead of a solution containing the aptide for STAT3 protein and nonaarginine.

### Comparative Example 2: Preparation of Nanoparticle Complex not Comprising Aptide for STAT3 Protein and Cell-Permeable Peptide, but Comprising Long-Chain and Short-Chain Phospholipids in Molar Ratio of 2:1

A nanoparticle complex was prepared in the same conditions and methods as described in Comparative Example 1, except that DMPC and DHPC were mixed in a molar ratio of 2:1.

### Comparative Example 3: Preparation of Nanoparticle Complex not Comprising Aptide for STAT3 Protein and Cell-Permeable Peptide, but Comprising Long-Chain and Short-Chain Phospholipids in Molar Ratio of 3:1

A nanoparticle complex was prepared in the same conditions and methods as described in Comparative Example 1, except that DMPC and DHPC were mixed in a molar ratio of 3:1.

### Experimental Example 1: Confirmation of Particle Size and Zeta Potential of Nanoparticle Complex

The particle size and zeta potential of the nanoparticle complexes prepared in Examples 1˜5 and Comparative Examples 1˜3 were confirmed by dynamic light scattering (DLS). The experiment was performed using a nanosizer ZS90 (Nanosizer ZS90, Malvern Instruments, Ltd., UK). In addition, the shape of the nanoparticle complex was confirmed by transmission electron microscopy using a JEM-3011 system (JEOL Ltd., Japan) at 300 kV. The mean diameter of the nanoparticle complex was calculated by measuring the size of at least 200 nanoparticle complexes using Image J software, version 1.49 (National Institute of Health, USA).

As a result, as shown in FIG. 1, as the molar ratio of the long-chain and short-chain phospholipids used in the preparation of the nanoparticle complex increased, the shape of the prepared nanoparticle complex changed from spherical to disc. This was not related to the presence or absence of the aptide for STAT3 protein and the cell-permeable peptide, but the nanoparticle complexes prepared in Examples 1˜3 were smaller, more numerous, and exhibited a clear discoid structure. On the other hand, the nanoparticle complexes prepared in Comparative Examples 1˜3 were easily aggregated. In particular, the nanoparticle complex prepared by mixing the long-chain and short-chain phospholipids in a molar ratio of 3:1 formed the smallest disc structure, and the mean diameter and thickness of the nanoparticle complex observed under a transmission electron microscope were 31.3±6.8 nm and 7.7±1.4 nm, respectively (FIG. 1).

It was observed by TEM that the nanoparticle complexes prepared in Examples 4 and 5 formed stable nanoparticles with a diameter of about 30 nm (FIGS. 2a and 2b).

In addition, as shown in FIGS. 3 and 4, it was confirmed through dynamic light scattering that the hydrodynamic diameter was about 20 to 30 nm (FIGS. 3 and 4).

On the other hand, the nanoparticle complexes prepared in Comparative Examples 1˜3 exhibited a negative charge on the surface, while the nanoparticle complexes prepared in Examples 1˜3 exhibited a positive charge (FIG. 5).

### Experimental Example 2: Confirmation of Skin Permeability of Nanoparticle Complex

### 2-1. Confirmation of Skin Permeability by Confocal Microscopy

The skin permeability by skin application was investigated using the nanoparticle complex prepared in Example 3, which was confirmed to have formed the smallest and most stable form in Experimental Example 1.

First, a nanoparticle complex ([FITC-APT]-LNC) was prepared in the same conditions and methods as described in Example 3, except that the aptide for STAT3 protein fused with FITC-labeled nonaarginine was used. At this time, the aptide (FITC-APT) for STAT3 protein fused with FITC-labeled nonaarginine was prepared as a control. Meanwhile, a psoriasis-induced murine animal model was prepared to remove the hair of the ear. The prepared [FITC-APT]-LNC and FITC-APT were applied to the ears of the psoriasis animal model in an amount of 50 μg, respectively. The ear tissue of the animal model was obtained as vertical cross sections 1, 6 or 12 hours after the application of [FITC-APT]-LNC and FITC-APT, respectively, and stored in a frozen state. The green fluorescence of FITC labeled to [FITC-APT]-LNC or FITC-APT in the obtained ear tissue was observed using a confocal microscope. The observed results were photographed and shown in FIG. 5.

As shown in FIG. 6, it was observed that the green fluorescence diffused into the skin tissue from 1 hour after the application of [FITC-APT]-LNC. The green fluorescence was observed in the upper dermis 6 and 12 hours after the application. However, the green fluorescence was not observed in the dermis when FITC-APT was applied. Part of this green fluorescence was observed in the hair follicles (FIG. 6).

Therefore, it was confirmed that the aptide for STAT3 protein fused with the cell-permeable peptide itself had no skin permeability, but the nanoparticle complex according to the present invention including this had skin permeability.

### 2-2. Confirmation of Skin Permeability by Two-Photon Microscopy

Next, the skin permeability of the nanoparticle complex according to the present invention was confirmed using a two-photon microscope. The experiment was performed in the same conditions and methods as described in Experimental Example 2-1, except that a two-photon microscope was used instead of a confocal microscope, and a normal animal model and a psoriasis animal model were used. At this time, a mode-locked tunable Ti:sapphire laser (Chameleon Ultra, Coherent) was used to adjust the wavelength of two-photon excitation of the two-photon microscope in the range of 690 to 1,020 nm. The 2D field of view was about 400×400 μm when 25× objective lens (CFI75 Apo LWD 25XW, NA1.1, Nikon) was used. At this time, the 3 different fluorescent signals CFP/SHG, GFP and RRP were detected simultaneously by 3 types of bandpass filters (FFO1-420/5, FF01-525/45, FF01-585/40, Semrock) and 3 types of photomultiplier tubes (R7518, Hamamatsu). The 2D images were processed with custom-written software.

As a result, as shown in FIG. 7, fluorescence signals were observed deeper and more uniformly in the ear tissue of the normal animal model applied with [FITC-APT]-LNC than in the ear tissue applied with FITC-APT. These fluorescence signals showed a typical polygonal structure of keratinocytes, and a second-harmonic generation (SHG) signal of collagen appeared as a red fibrous structure that is a marker of the dermis layer in the tissue deeper than about 20 μm from the epidermis. In particular, the fluorescence signals of [FITC-APT]-LNC applied on the skin were observed in the upper dermis, whereas most of FITC-APT remained in the stratum corneum (FIG. 7).

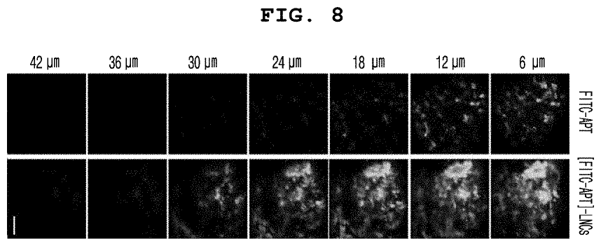

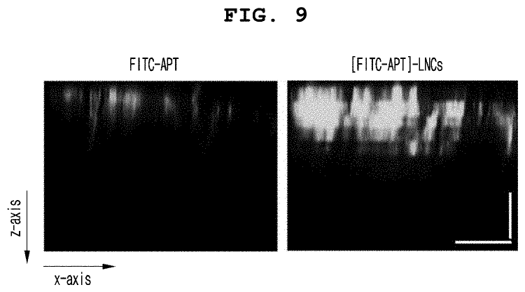

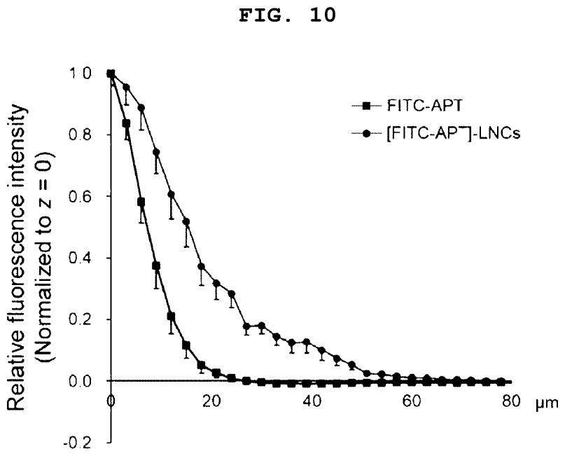

As shown in FIGS. 8 and 9, in the psoriasis animal model, the cell polarity disappeared due to the abnormal proliferation of keratinocytes, and cells with different structures appeared on the skin surface. The fluorescence signals of [FITC-APT]-LNC were observed at a depth of about 40 to 60 m in the skin, while the fluorescence signals of FITC-APT were observed at a depth of about 10 to 20 μm in the skin (FIGS. 8 and 9). In addition, the fluorescence signals of [FITC-APT]-LNC and FITC-APT were graphed according to the depth and intensity based on the above results. As a result, as shown in FIG. 10, the fluorescence signal was strong when [FITC-APT]-LNC was applied, which was observed even at a depth of up to 60 m (FIG. 10).

### Experimental Example 3: Confirmation of Skin Cell Permeability According to Treatment

Concentration of Nanoparticle Complex First, HaCaT cells (ratinocytes) were cultured in DMEM supplemented with 10% fetal bovine serum and 1% penicillin and streptomycin. The cultured cells were distributed in 12-well plates at the density of 1×10⁵ cells/well, and cultured in under the conditions of 5% CO₂ at 37° C. The cultured cells were treated with the [FITC-APT]-LNC prepared in Experimental Example 2 at the concentration of 1, 5 or 10 M. At this time, as a control, the negative control group with no treatment was used. The cell permeability of [FITC-APT]-LNC to HaCaT cells was confirmed using a flow cytometer 90 minutes after the treatment of [FITC-APT]-LNC

As a result, as shown in FIG. 11, the fluorescence intensity was increased in proportion to the concentration of [FITC-APT]-LNC (FIG. 11).

### Experimental Example 4: Confirmation of Skin Cell Permeability According to Treatment Time of Nanoparticle Complex

The skin cell permeability of the nanoparticle complex according to the treatment time was investigated in the same conditions and methods as described in Experimental Example 3 except that the cell permeability to HaCaT cells was confirmed using a flow cytometer 0.25, 0.5, 1, 2, 4 or 8 hours after the treatment of the [FITC-APT]-LNC prepared in Experimental Example 2 at the concentration of 10 M.

As a result, as shown in FIG. 12, the fluorescence intensity was increased in proportion to the treatment time of [FITC-APT]-LNC, and the intensity was strongest 2 to 4 hours after the treatment (FIG. 12).

### Experimental Example 5: Confirmation of Intracellular Delivery of Nanoparticle Complex

HaCaT Cells (Keratinocytes) and NIH3T3 Cells (Fibroblasts) were Treated with the [FITC-APT]-LNC prepared in Experimental Example 2 at the concentration of 10 M. The experiment was performed in the same conditions and methods as described in Experimental Example 3, except that the observation was conducted using a microscope after 6 hours. At this time, the group treated with PBS was used as a control group. The cells treated with [FITC-APT]-LNC were observed under a confocal microscope, and the results are shown in FIG. 13.

As shown in FIG. 13, it was confirmed that [FITC-APT]-LNC penetrated the cells and was located in the cytoplasm and nucleus (FIG. 13).

### Experimental Example 6: Confirmation of Inhibitory Effect of Nanoparticle Complex on Inflammation Caused by Psoriasis

The nanoparticle complex prepared in Example 3 was applied on the skin of a psoriasis animal model and the inhibitory effect of the complex on inflammation caused by psoriasis was investigated.

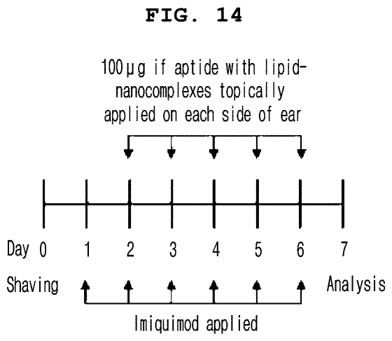

First, the hair of the ear of the mouse was removed, and imiquimod was continuously treated at the concentration of 20 mg/cm² for 6 days from the next day to induce psoriasis. On the other hand, on the 2^{nd} to 6^{th} day of the treatment of imiquimod, the nanoparticle complex of Example 3 or Comparative Example 3 was applied in a total amount of 100 μg, 50 μg per time. On the day when imiquimod and the treatment drug were treated together, the treatment drug was applied 4 hours before and after the application of imiquimod to prevent the interaction between the drugs. At this time, the groups treated with Vaseline and DW were used as the negative control, and the group applied with clobetasol propionate (CLQ) at the concentration of 20 mg/cm² was used as the positive control. The application of the nanoparticle complex was completed by day 6, and the inhibitory effect on inflammation was confirmed using the animal model on day 7 (FIG. 14). Particularly, the inhibitory effect on inflammation was confirmed by measuring the thickness of the ear of the animal model, the punch biopsy weight, tissue H&E test, ELISA after tissue crushing, and the extracted spleen weight. All the experiments were performed by the conventional methods.

As a result, as shown in FIGS. 15a-15c, the ear thickness, PSAI score and ear punch biopsy weight were significantly reduced in the animal model treated with the nanoparticle complex of Example 3 compared to the negative control group (FIGS. 15a˜15c). As shown in FIG. 16, it was confirmed by visual observation that psoriasis was ameliorated in the animal model treated with the nanoparticle complex of Example 3, consistent with the above results (FIG. 16).

As a result of histological analysis, as shown in FIG. 17a, the animal model treated with distilled water or the nanoparticle complex of Comparative Example 3 showed hyperplasia of the epidermis and infiltration of inflammatory cells, whereas such pathological properties were significantly reduced in the animal model treated with the nanoparticle complex of Example 3 (FIG. 17a). As shown in FIG. 17b, the productions of IL-17, IL-12/23p40 and IL-1β, the psoriasis-related cytokines, were significantly reduced in the animal model treated with the nanoparticle complex of Example 3 (FIG. 17b).

As shown in FIGS. 18a and 18b, the size and weight of the spleen were increased in all the experimental groups except the CLQ-treated group, but the spleen of the group treated with CLQ was shrivelled. It is presumed that the complex affected the systemic immune system. On the other hand, the topical treatment effect of the nanoparticle complex of Example 3 on psoriasis was similar to that of the positive control CLQ (FIGS. 18a and 18b).

Therefore, it was confirmed that the nanoparticle complex according to the present invention effectively alleviated the inflammation caused by psoriasis without side effects even when the complex was applied on the skin

### Experimental Example 7: Confirmation of Anti-Fibrosis Effect of Nanoparticle Complex

The expression changes in of Col1a1 (collagen 1a1) and αSMA (α-smooth muscle actin), the fibrosis-related genes, by the nanoparticle complex of Example 3 were confirmed by the following method.

First, NIH3T3 cells, a mouse embryonic fibroblast line, were prepared using DMEM supplemented with 10% fetal bovine serum (FBS) and 1% antibiotics. The prepared cells were distributed in 12-well plates at the density of 1.5×10⁴ cells/well, which were cultured for overnight. After replacing the medium with the FBS-free medium, the cells were further cultured for 24 hours, and treated with TGF-β at the concentration of 10 ng/m. Eighteen hours later, the nanoparticle complex of Example 3 (10 μM) was treated thereto, and the cells were further cultured for 6 hours.

At this time, the lipid nanoparticle complex (APTscr-9R) prepared in the same manner as described in Example 3, except that an aptide composed of the amino acid sequence represented by SEQ. ID. NO: 9 was used, was used as a control. Upon completion of the culture, only cells were obtained, and real-time PCR was performed by the conventional method using the primers listed in Table 1 below to confirm the expression changes of Col1a1 and αSMA genes. The results were normalized based on the expression level of GAPDH gene and shown in FIG. 19.

TABLE 1

| Name | Sequence (5′→3′) | SEQ. ID. NO. |
|---|---|---|
| Col1a1_forward | TAG GCC ATT GTG TAT GCA GC | SEQ. ID. NO: 11 |
| Col1a1_reverse | ACA TGT TCA GCT TTG TGG ACC | SEQ. ID. NO: 12 |
| αSMA_forward | GTC CCA GAC ATC AGG GAG TAA | SEQ. ID. NO: 13 |
| αSMA_reverse | TCG GAT ACT TCA GCG TCA GGA | SEQ. ID. NO: 14 |
| GAPDH_forward | TTC ACC ACC ATG GAG AAG GC | SEQ. ID. NO: 15 |
| GAPDH_reverse | GGC ATG GAC TGT GGT CAT GA | SEQ. ID. NO: 16 |

As shown in FIG. 19, the gene expressions of Col1a1 and αSMA, known as fibrosis-inducing cytokines, were increased about 2 times by TGF-β, but the expressions were decreased to the normal levels by the treatment of the lipid nanoparticle complex according to the present invention (FIG. 19).

These results were consistent with the previous report that STAT3 protein inhibitors can inhibit fibrosis in the kidney, liver, or lung (Kidney International, 2010; Clin Cancer Res, 2017; EMBO Mol Med, 2012), from which it was confirmed that the lipid nano complex according to the present invention can also be used for the treatment of fibrosis.

### Experimental Example 8: Confirmation of Colloidal Stability of Nanoparticle Complex

The colloidal stability of the lipid nanoparticle complex prepared in Example 3 was confirmed by the following method.

First, DLS analysis was performed in the same conditions and methods as described in Example 1 using the lipid nanoparticle complex prepared in Example 3. DLS analysis was performed until 15 days after the preparation, and the size of the identified lipid nanoparticle complex are shown in FIG. 20b. At this time, the lipid nanoparticle complex prepared in Comparative Example 3 was used as a control

As shown in FIG. 20a, the lipid nanoparticle complex prepared in Comparative Example 3 (left) formed aggregates after 6 hours of the preparation, but the lipid nanoparticle complex prepared in Example 3 (right) did not form aggregates (FIG. 20a). As shown in FIG. 20b, the lipid nanoparticle complex prepared in Example 3 maintained a discoid structure of about 30 nm in diameter even after 15 days of the preparation (FIG. 20b).

### Experimental Example 9: Confirmation of Suppressive Effect of Nanoparticle Complex on the M2 Polarization of Macrophages and Epithelial-to-Myofibroblast Differentiation

Inhibiting STAT3 activation of IL-10 stimulated RAW264.7 cells by treating the lipid nanoparticle complex prepared in Example 3 effectively suppressed the macrophage polarization.

To perform FACS analysis, mouse macrophage cell line (RAW264.7) was seeded in a 6-well plate at 2×10⁵ cells/well density and incubated overnight for stabilization. In order to induce M2-type polarization, the samples were stimulated with IL-10 for 48 h in a 37° C.-maintained, 5% CO2 incubator. PBS, APTscr-9R(20 μM), and APTstat3-9R(20 μM) were simultaneously accordingly added for co-treatment. After the incubation, the specimens were collected and incubated for 30 min with 10% BSA in PBS to block non-specific binding of antibodies. After blocking, the samples were washed with three changes of 1×PBS for 5 min each. The collected specimens were resuspended in a 1 mL of separation buffer. The cells were collected in the filter tubes with strainer (70 m). After buffer removal, direct immunostaining was performed using phycoerythrin-cojugated CD163 monoclonal primary antibody (Invitrogen, Waltham, Mass., USA) at 0.25 g/sample with 90 min incubation at 4° C. Upon the incubation, the cells were once again washed with three changes of 1×PBS for 5 min. After centrifugation (1350 rpm, 5 min), the buffer was removed and fresh PBS containing 1% PFA was used to prepare final samples. The samples (20,000 cells) were analyzed using a LSR Fortessa™ high-performance multi-parameter flow cytometer (BD Bioscience, San Jose, Calif., USA) processed with FlowJo software (Tree Star Inc., San Carlos, Calif., USA).

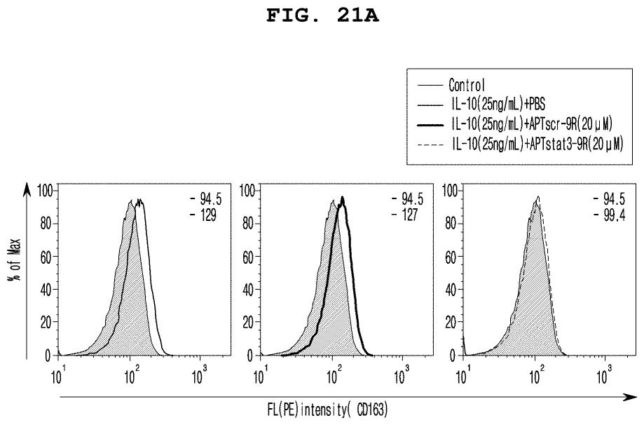

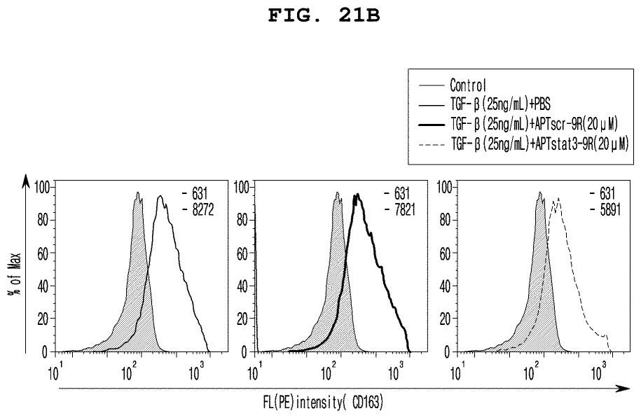

From FACS analysis, expression of M2 macrophage specific marker, CD163 was not upregulated when APTstat3-9R was treated, while PBS and control aptide(APTscr-9R, SEQ ID NO:9) treated samples showed approximately 30˜40% increase in CD163 level when they were stimulated with IL-10 (FIG. 21a).

Furthermore, FACS analysis using mesenchymal filament marker, vimentin, showed similar results. TGF-β is a well-known STAT3 activator and proliferation and differentiation mediator in endothelial mesenchymal transition. Thus, while PBS and APTscr-9R treated MLE12 cells showed vastly increased vimentin marker levels after the stimulation with TGF-0, APTstat3-9R treated sample exhibited fairly decreased vimentin level (approximately 29% reduction compared to PBS treated group) (FIG. 21b).

### Experimental Example 10: Confirmation of Lung Epithelial Cell Uptake and Pulmonary Surfactant Barrier Penetration of Nanoparticle Complex

In order to achieve successful pulmonary admission, the inhaled or injected therapeutic agents will need to primarily pass by airway branches and infiltrate the first encountering biologic barrier, pulmonary surfactant layer. Surfactant layer penetrated peptides should be able to enter the lung epithelial cells. Lung surfactant permeation validation was performed in bleomycin-induced pulmonary fibrosis mice.

Pulmonary fibrosis mice model was induced with single bleomycin lung instillation. Bleomycin sulfate (Tokyo Chemical Industry Co., Tokyo, Japan) was dissolved in 1×PBS and each subject received weight-adjusted dosages of bleomycin at a dose of 1 mg/kg via intratracheal instillation using 20 gauge polyurethane catheter (BRAUN, Hessen, Germany) while being anesthetized with 30 mg/kg of tiletamine/zolazepam and 10 mg/kg of xylazine application at day 0. At the same time, control group mice received an equal volume (100 μL) of 1×PBS. In the course of all instillations throughout the experiments, the nostril was occluded with a thumb, driving the mouse to breathe with the mouth to affirm the deposition into the lung. The thumb was released after two breathings were finished.

Disease-induced animals were prepared with single instillation of bleomycin (day 0). On day 14, PBS, FITC conjugated APTstat3-9R, and FITC conjugated APTstat3-9R-DLNPs were respectively treated to the subjects via intratracheal instillation. After 4 h, whole-body perfusion using ice cold sterile PBS was completed to blench the organs in order to avoid biased fluorescence detection from remaining blood. Successively, 3 times of lung flushing through trachea cannulation using wash buffer (1×PBS) was performed to remove free aptides (w/ and w/o lipid formulation) within the air passageway. Finally, mice organs (liver, spleen, kidney, lung, and heart) were collected and immediately analyzed under IVIS Spectrum in vivo Imaging System (PerkinElmer, Waltham, Mass., USA).

In vitro cell uptake of FITC-APTstat3-9R and FITC-APTstat3-9R-DLNPs was determined by confocal microscopy imaging and FACS analysis. For confocal imaging, MLE12 cells were seeded on sterile cover glass in 12-well plate at 5×10⁵ cells/well density followed by overnight incubation. The cells were treated with PBS, FITC-APTstat3-9R (20 μM), or FITC-APTstat3-9R-DLNPs (20 μM) for 4 h. The samples were washed twice with 1×PBS to discard free aptides. Subsequent sample preparation and analysis were described previously in in vitro macrophage polarization analysis section. For FACS experiment, MLE 12 cells were seeded in a 6-well plate at 3×10⁵ cells/well density and incubated overnight for stabilization. The samples were treated with PBS, FITC-APTstat3-9R (20 μM), or FITC-APTstat3-9R-DLNPs (20 μM) for 4 h, washed, and collected using trypsin. The following steps were previously detailed in in vitro macrophage polarization analysis.

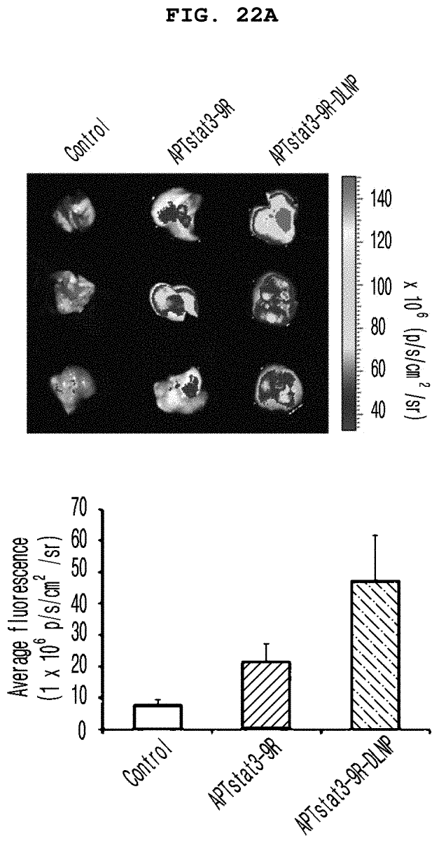

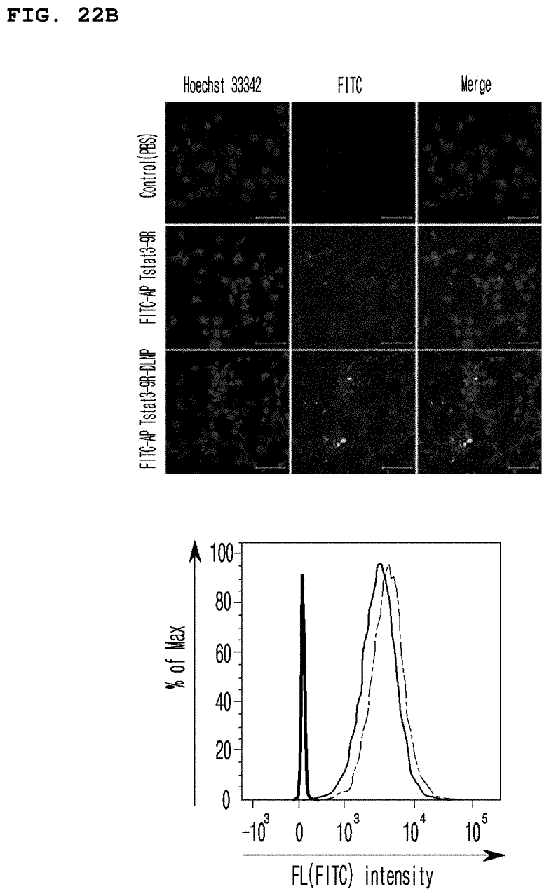

Lung sections after intratracheal treatment of FITC-APTstat3-9R-DLNPs revealed that significantly increased amount of APTstat3-9R-DLNPs were internalized into the lung parenchyma. In terms of fluorescence intensity, nearly 2.2-fold increased amount of APTstat3-9R was detected when they were encased in lipid nanoparticles (FIG. 22a).

In addition, it was clear in confocal images that both APTstat3-9R and APTstat3-9R-DLNPs were uptaken into MLE 12 lung epithelial cells. Compared by quantification using FACS evaluation, approximately 11% of increase in FITC-labeled APTstat3-9R penetration was observed when aptide was encased in DLNP (FIG. 22b).

### Experimental Example 11: Confirmation of Attenuating Effect of Nanoparticle Complex on Pulmonary Fibrosis In Vivo Model

The in vivo efficacy of APTstat3-9R-DLNPs was assessed by intratracheal instillation delivery (50 g, 10 wt %) in bleomycin-induced pulmonary fibrosis mice model of experimental example 10.

Starting a day after bleomycin administration, PBS (100 μL), APTscr-9R-DLNPs (50 g in 100 μL of PBS, 10 wt %), or APTstat3-9R-DLNPs (50 g in 100 μL of PBS, 10 wt %) were intratracheally instilled every 2 days for 14 days. On day 14, subjects were sacrificed, and blood serum, spleen, and lung specimen were collected for further analysis.

Daily body-weight was measured prior to instillation of PBS and aptides to avoid biased body weight results. Weight of wet left-side lung samples was measured upon the sacrifice, and the very samples were completely dried with freeze-dry vacuum. Fully dehydrated lung samples were collected and their weight was measured.

Starting a day after bleomycin instillation, APTstat3-9R-DLNPs administration was carried out every other day for 14-day duration (FIG. 23a). Unintended body weight loss being a stated symptom of pulmonary fibrosis, daily whole body weight was monitored for 14-day time course. Mice in disease-free (Control) group showed gradual body weight increase from day 1 while all other groups (bleomycin+PBS, bleomycin+APTscr-9R-DLNPs, bleomycin+APTstat3-9R-DLNPs) displayed slight drop in body weight after bleomycin administration. Only APTstat3-9R-DLNPs treated mice exhibited body weight recover at the time of sacrifice while bleomycin+PBS and bleomycin+APTscr-9R-DLNPs treated groups showed continuous drop in body weight (FIG. 23b and FIG. 23c).

In addition, several anatomical indexes of pulmonary fibrosis (lung weight, lung (mg)/body (g) weight ratio, and lung wet-to-dry ratio) were examined. Typically, lung weight and lung/body (L/D) weight ratio are increased in pulmonary fibrosis induced lungs due to exaggerated ECM accumulation. Bleomycin instillation clearly induced pulmonary fibrosis-like lung condition characterized by significant lung weight and L/D weight ratio increase in bleomycin+PBS treated group. Remarkably, lung weight of bleomycin+APTstat3-9R-DLNPs treated mice retained their lung weight comparative to the control groups and only a minimal increase of L/D weight ratio was observed. Interestingly, bleomycin+APTscr-9R-DLNPs treated groups exhibited the most severely elevated lung weight and L/D weight ratio (FIG. 23d and FIG. 23e).

Escalation of lung wet-to-dry (W/D) ratio is often looked into when diagnosing lung complications because damaged lung has dysregulated lung permeability, resulting in pulmonary edema described by excess lung water accumulation. Insignificant W/D ratio increase was observed for bleomycin+PBS group whereas bleomycin+APTscr-9R-DLNPs group exhibited noteworthy W/D ratio rise. Negligible W/D ratio increase was seen which matched with the outcomes from lung weight and L/D ratio results (FIG. 23f).

### Experimental Example 12: Histological Analysis of Experimental Animal Models

Histological analysis of tissue sections incised to 4 m thickness was performed with Hematoxylin & Eosin (H&E) to visualize pathologic changes and Sirius red & Fast green staining to envision collagen deposition in the tissue.

Right lung samples were immediately washed with 1×PBS and fixed with 4% PFA for 12 h followed by tissue dehydration, which were processed to paraffin blocks and sectioned (4 m thickness) with Leica CM 1850 microtome (Leica, Wetzlar, Germany) on the HistoBond glass slides (Marienfeld Superior, Lauda-Konigshofen, Germany). Prepared tissue samples were deparaffinized with histological grade xylene (Sigma-Aldrich, St. Louis, Mo., USA) and rehydrated for Hematoxylin & Eosin (H&E) and Sirius red & Fast green staining. Stained samples were washed with three changes of distilled water to remove excess dye followed by serial dehydration in two changes of 95% ethanol and 100% ethanol. The stained tissues were cleared in two changes of xylene for 3 min each and the samples were mounted with Permount mounting medium (Fisher Scientific, Fair Lawn, N.J., USA). Histology of mounted lung samples was visualized using an inverted microscope (Eclipse Ti2; Nikon, Tokyo, Japan). Pulmonary fibrosis score of lung samples was calculated by objective Ashcroft scoring system by a third party. Collagen quantification of Sirius red & Fast green stained samples was analyzed using a dye extraction buffer in Sirius Red/Fast Green collagen Staining kit (Chondrex Inc., Redmond, Wash., USA) with appropriate absorbance compensation calculation ([OD₅₄₀−(OD₆₀₅×0.291)]/0.0378).

H&E stained tissue images (scale: 100 m) revealed tremendously increased tissue density with distinctively thickened alveolar walls and observable distortion of lung structures (pink, and air spaces in white), goblet cell hyperplasia (blue-purple), and infiltration of inflammatory cells (blue-purple) for PBS and APTscr-9R-DLNPs treated groups. For APTstat3-9R-DLNPs treated groups, minimal to moderate alveolar and bronchiole wall thickening was observed but tissue density, infiltrated inflammatory foci were mildly maintained similar to disease-free group samples (FIG. 24a). Severity of disease was calculated using objective Ashcroft scoring system (FIG. 24c). In Sirius red & Fast green staining, collagen is characterized by red color and non-collagenous protein is stained in green. Very small amount of collagen deposition was detected for disease-free and APTstat3-9R-DLNPs treated groups however, significant collagen accumulation was seen across the field for PBS and APTscr-9R-DLNPs treated groups (FIG. 24b). Collagen amount was quantified using dye extraction buffer with appropriate absorbance compensation (FIG. 24d).

### Experimental Example 13: Immunohistochemical Analysis of Lung Tissues

Additionally, IHC staining analysis was carried out to visualize distinct pulmonary fibrosis markers (CD163, pSTAT3, CD20, and mast cell). CD163 is a well-known M2 macrophage specific marker therefore as a stretch from previous macrophage polarization experiment, identification of CD163 is pivotal in pulmonary fibrosis IHC analysis.

Lung sections were deparaffinized and rehydrated following the same protocol from the histological analysis. Toluidine blue working solution was used to stain mast cells. Toluidine blue working solution was prepared by mixing 5 mL of Toluidine blue O stock (Sigma-Aldrich, St. Louis, Mo., USA) dissolved in 70% ethanol and 45 mL of 1% sodium chloride solution (pH 2.5). The slides were immersed in working solution for 3 min then washed with three changes of distilled water to remove excess staining dye. Dehydration and mounting were carried out following the same procedures at histological analysis section. For immunohistochemistry, antigen unmasking was done by immersing slides in 10 mM sodium citrate buffer (pH 6.0) and maintaining the buffer temperature just below the boiling point for 10 min. Subsequently, the slides were cooled on the benchtop for 30 min. After, the slides were immersed in 3% hydrogen peroxide in distilled water for 10 min to quench background peroxidase activity. After washing with wash buffer (1×TBS/0.1% Tween-20) the area around the samples was wiped and hydrophobic pen (ThermoFisher Scientific, Waltham, Mass., USA) was used to draw boundaries around the samples to avoid reagent waste in the following steps. Blocking with blocking buffer (1×TBS, 3% BSA, and 0.2% Triton X-100) was performed for 1 h to prevent non-specific binding. Blocking buffer was removed, then primary antibody in diluent (1×TBS/1% BSA), anti-phospho-STAT3 (1:100; abeam, Cambridge, UK), anti-CD20 (1:100; abeam, Cambridge, UK), or anti-CD163 (1:500; abeam, Cambridge, UK) were added to each slide followed by overnight incubation at 4° C. in a humidified chamber. The antibodies solution was washed with three changes of wash buffer, then secondary antibody in diluent, HRP-conjugated goat anti-rabbit IgG (1:2000; abeam, Cambridge, UK) was added to each slide and incubated for 1 h in room temperature. The secondary antibody solution was also removed with washing step (three changes of wash buffer). After, 3, 3-diaminobenzidine (DAB) substrate (abeam, Cambridge, UK) was added to the slides for 10 min to visualize chromogenic staining in dark brown color. As a final step, slides were counterstained using toluidine blue for 30-60 sec followed by washing with two changes of distilled water. Dehydration, sample mounting, and microscopy imaging were performed according to the same protocol from the histological analysis. Stained mast cells and marker-positive areas were quantified with at least 10 tissue samples using ImageJ software.

From quantification of CD163 expressed area, there were obvious increase in M2 macrophage marker in PBS and APTscr-9R-DLNPs treated groups but samples from APTstat3-9R-DLNPs treated group did not induce M2 polarization of macrophages (FIG. 25a and FIG. 25b). Similar to CD163 IHC result, activated from of STAT3 (pSTAT3) was relatively abundant in PBS and APTscr-9R-DLNPs treated tissues while control group showed only basal level of phosphor-STAT3 expression. APTstat3-9R-DLNPs treated subjects exhibited slight phosphor-STAT3 manifestation but significant amount of inhibition was observed. (FIG. 25a and FIG. 25c). Expression of CD20 was abundantly observed for PBS and APTscr-9R-DLNPs treated groups but almost no or only nominal area was detected to be CD20-positive for disease-free and APTstat3-9R-DLNPs treated tissues, respectively (FIG. 25a and FIG. 25d). It has been known that increased populations of mast cell is distinctive in the lungs of pulmonary fibrosis patients. Toluidine blue staining was practiced to stain mast cells of all samples and from cell counting using ImageJ software, approximately 10-fold increase in mast cells number compared to control was observed for PBS and APTscr-9R-DLNPs treated samples while APTstat3-9R-DLNPs treated samples ended up in mere 2-fold increase (FIG. 25a and FIG. 25e).

### Experimental Example 14: Toxicological Evaluation of Nanoparticle Complex

Extensive toxicological parameters were evaluated to certify the safety of APTstat3-9R-DLNPs.

The blood biochemical analysis of AST, ALT, BUN, CRE, and GLU was performed with collected serum from the animal model using a clinical chemistry analyzer (AU680; Beckman Coulter, Brea, Calif., USA) and an electrolyte analyzer (M744, SIEMENS, Erlangen, Germany). The pictures of arranged spleens were taken and weighed.

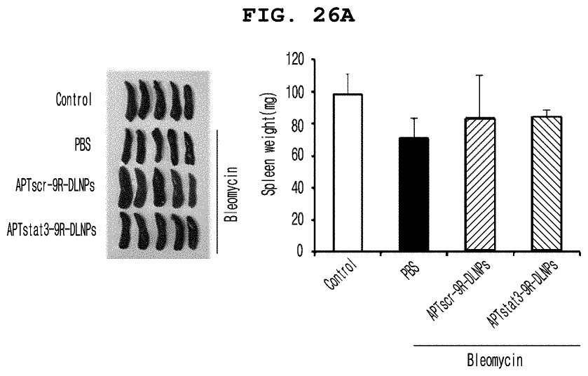

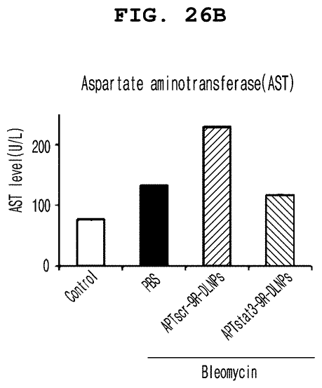

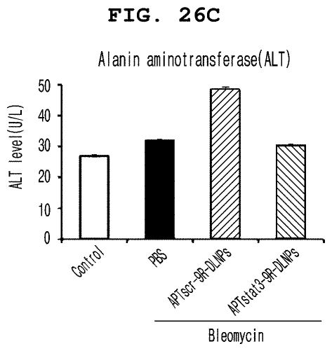

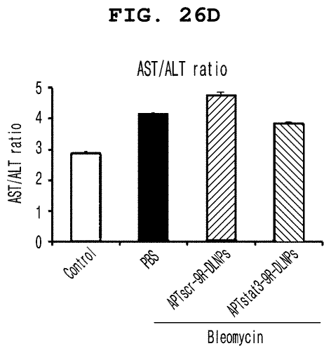

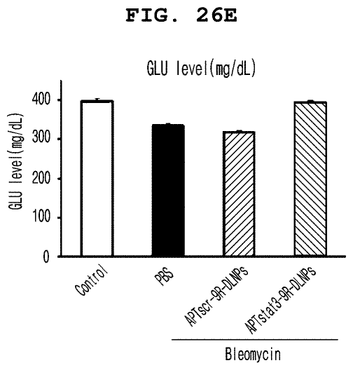

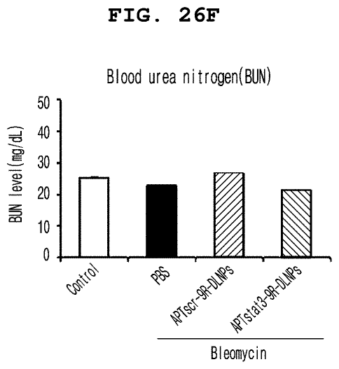

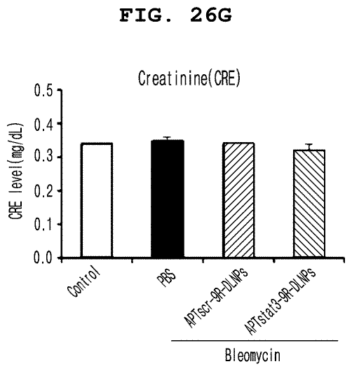

Administration of bleomycin exhibited splenic toxicity in fair degree; however, aptide administrated mice subjects showed somewhat recovered spleen size and weight (FIG. 26a). In addition, serum biochemical analysis of Aspartate aminotransferase (AST), Alanine aminotransferase (ALT), Glucose (GLU), Blood urea nitrogen (BUN), and Creatinine (CRE) was performed. AST and ALT are characteristic indicators of systemic hepatotoxicity. APTstat3-9R-DLNPs treated mice demonstrated only basal levels of inflammatory enzymes while APTscr-9R-DLNPs administration showed 2 to 3-fold elevation in ALT and AST, separately (FIG. 26b and FIG. 26c). Notably, APTstat3-9R-DLNPs treated group exhibited the lowest AST/ALT ratio among bleomycin-treated subjects (FIG. 26d). Non-diabetic hypoglycemia was observed in the PBS and APTscr-9R-DLNPs treated group mice (FIG. 26e). BUN and CRE are indexes of acute kidney failure from possible inflammation, medication, and toxicity. No meaningful kidney damage was observed for all tested groups (FIG. 26f and FIG. 26g).

What is claimed is:

1. A lipid nanoparticle complex comprising an aptide fused with a cell penetrating material.

2. The lipid nanoparticle complex according to claim 1, wherein the lipid nanoparticle complex comprises long-chain and short-chain phospholipids.

3. The lipid nanoparticle complex according to claim 2, wherein the long-chain and short-chain phospholipids are included in a molar ratio of 0.5˜7:1.

4. The lipid nanoparticle complex according to claim 2, wherein the long-chain phospholipid is any one or more selected from the group consisting of 1,2-dilauroyl-sn-glycero-3-phosphocholine(12:0 PC, DLPC), 1,2-ditridecanoyl-sn-glycero-3-phophocholine (13:0 PC), 1,2-dimyristoyl-sn-glycero-3-phosphocholine (14:0 PC, DMPC), 1,2-dipentadecanoyl-sn-glycero-3-phophocholine (15:0 PC), 1,2-dipalmitoyl-sn-glycero-3-phosphocholine (16:0 PC, DPPC), 1,2-diphytanoyl-sn-glycero-3-phosphocholine (4ME 16:0 PC), 1,2-diheptadecanoyl-sn-glycero-3-phophocholine (17:0 PC), 1,2-distearoyl-sn-glycero-3-phosphocholine (18:0 PC, DSPC), 1,2-dinonadecanoyl-sn-glycero-3-phophocholine (19:0 PC), 1,2-diarachidoyl-sn-glycero-phosphocholine (20:0 PC), 1,2-dihenarachidoyl-sn-glycero-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-palmitoyl-2-oleoyl-sn-glycero-3-phosphocholine (POPC) and 1-palmitoyl-2-stearoyl-sn-glycero-3-phosphocholine (PSPC).

5. The lipid nanoparticle complex according to claim 2, wherein the short-chain phospholipid is any one or more selected from the group consisting of 1,2-dipropionyl-sn-glycero-3-phosphocholine (3:0 PC), 1,2-dibutyryl-sn-glycero-3-phosphocholine (4:0 PC), 1,2-dipentanoyl-sn-glycero-3-phosphocholine (5:0 PC), 1,2-dihexanoyl-sn-glycero-3-phosphocholine (6:0 PC, DHPC), 1,2-diheptanoyl-sn-glycero-3-phosphocholine (7:0 PC, DHPC) and 1,2-dioctanoyl-sn-glycero-3-phosphocholine (8:0 PC).

6. The lipid nanoparticle complex according to claim 1, wherein the lipid nanoparticle complex has a discoid structure.

7. The lipid nanoparticle complex according to claim 1, wherein the cell penetrating material is at least one selected from the group consisting of peptides and compounds.

8. The lipid nanoparticle complex according to claim 1, wherein the lipid nanoparticle complex has a diameter of 10˜500 nm.

9. The lipid nanoparticle complex according to claim 1, wherein the aptide fused with the cell penetrating material is included at the concentration of 0.2 to 30 weight % by the total weight of the lipid nanoparticle complex.

10. The lipid nanoparticle complex according to claim 1, wherein the colloidal stability of the lipid nanoparticle complex is maintained for more than 1 hour in an aqueous solution.

11. A method for treating or ameliorating inflammatory skin disease comprising a step of administering the lipid nanoparticle complex of claim 1 to a subject in need thereof.

12. The method according to claim 11, wherein the inflammatory skin disease is psoriasis or atopy.

13. The method according to claim 11, wherein the
lipid nanoparticle complex is applied to a skin of a subject.

14. A method for treating or ameliorating fibrosis comprising a step of administering the lipid nanoparticle complex of claim 1 to a subject in need thereof.

15. The method according to claim 14, wherein the fibrosis is selected from the group consisting of pulmonary fibrosis, renal fibrosis and liver fibrosis.

16. The method according to claim 14, wherein the lipid nanoparticle complex is administered by an intratracheal instillation.
